(19) United States
(12) Patent Application Publication
(10) Pub. No.: US 2025/0246314 A1
(43) Pub. Date: Jul. 31, 2025

(54) METHODS FOR THE DETECTION AND TREATMENT OF PANCREATIC DUCTAL ADENOCARCINOMA

(71) Applicant: Board of Regents, The University of Texas System, Austin, TX (US)

(72) Inventors: Samir HANASH, Houston, TX (US); Johannes Francois FAHRMANN, Houston, TX (US); Ehsan IRAJIZAD, Houston, TX (US); Jennifer Bolin DENNISON, Houston, TX (US); Eunice N. MURAGE, Missouri City, TX (US); Ranran WU, Houston, TX (US)

(21) Appl. No.: 19/074,016

(22) Filed: Mar. 7, 2025

Related U.S. Application Data
(63) Continuation of application No. PCT/US2023/073951, filed on Sep. 12, 2023.
(60) Provisional application No. 63/375,328, filed on Sep. 12, 2022.

(51) Int. Cl. G16H 50/30; G01N 30/02; G01N 30/72; G01N 30/86; G01N 30/88; G01N 33/68; G06N 20/00; G16B 40/10; G16H 50/20; G16H 50/70

(52) U.S. Cl. CPC G16H 50/30; G01N 30/7233; G01N 30/8696; G01N 33/6851; G01N 33/6893; G06N 20/00; G16B 40/10; G16H 50/20; G16H 50/70; G01N 2030/027; G01N 2030/8831; G01N 2030/8836; G01N 2030/884; G01N 2333/46

(57) ABSTRACT

A novel 3-marker microbial-related metabolite panel (3MMP), consisting of or comprising TMAO, indoleacrylic acid, and an indole derivative, and a novel 5-marker non-microbial metabolite panel, consisting of or comprising cholesterol glucuronide, 2-hydroxyglutarate, galactosamine, glucose, and erythritol, capable of assessing a 5-year risk of pancreatic cancer is described.

This application is a bypass continuation of International Application No. PCT/US2023/073951, filed Sep. 12, 2023, which claims the benefit of priority to U.S. Provisional Application No. 63/375,328, filed Sep. 12, 2022, the disclosures of each are hereby incorporated by reference as if written herein in their entireties.

This application was made with government support under U01 CA196403, U01 CA200468, and P50-CA221707, awarded by the National Cancer Institute and the National Institutes of Health. The government has certain rights in the invention.

Disclosed herein are methods and related kits for detection of early-stage pancreatic ductal adenocarcinoma. Also provided are methods for treating a patient susceptible, or suspected of being susceptible, to pancreatic ductal adenocarcinoma.

Pancreatic cancer (pancreatic ductal adenocarcinoma, PDAC) is the third most common cause of cancer deaths in the United States and is projected to become the second-leading cause by 2040. Surgical resection of localized disease represents the greatest chance for curative therapy. Unfortunately, only a minority (15-20%) of patients present with surgically resectable disease.

The low incidence of pancreatic cancer in the average-risk population (˜8-12 per 100,000) makes it challenging to implement effective screening programs for pancreatic cancer, and the United States Preventative Services Task Force (USPSFT) currently recommends against screening of pancreatic cancer in the general population. However, the USPSFT recognizes that screening in persons who are at an increased risk may be warranted. There remains an opportunity to develop blood-based signatures that can identify individuals at increased risk who would benefit from screening.

The microbiota is a complex ecosystem integral to human health. Microbial diversity is site-specific and varies depending on the location in the body. Increasing evidence indicates that alterations in the microbiome are associated with cancer risk, including pancreatic cancer. Studies suggest that loss of microbial diversity and community stability, along with increases in pathogenic microbes, increase cancer susceptibility. In the context of pancreatic cancer, changes in the microbiome have been linked to altering the tumor microenvironment and tumor immunophenotype, as well as response to chemotherapy and immunotherapy and long-term survival in patients with resected pancreatic adenocarcinoma (PDAC).

The microbiome is strongly associated with changes in metabolism that can perpetuate inflammation and increase an individual's risk of developing cancer, including pancreatic cancer. Microbiome-related metabolites include short-chain fatty acids, butyrate and acetate, secondary bile acids, indole-derivatives, cadaverine, trimethylamine N-oxide (TMAO), and lipopolysaccharides. Elevated serum levels of TMAO, a gut microbiota-derived pro-inflammatory metabolite, were reported to be associated with increased risk of pancreatic cancer in the Shanghai Cohort Study and the Singapore Chinese Health Study. TMAO has also been reported to be elevated in plasmas collected ≥2 years before a diagnosis of pancreatic cancer compared to matched controls. Plasma levels of indoleacrylic acid and indole-3-acetate have been shown to differentiate newly diagnosed pancreatic cancer cases from controls.

Accordingly, a need exists for a method or test to aid the detection of pancreatic cancer at an early stage. A novel 3-marker microbial-related metabolite panel (3MMP), consisting of or comprising TMAO, indoleacrylic acid, and an indole derivative, and a novel 5-marker non-microbial metabolite panel, consisting of or comprising cholesterol glucuronide, 2-hydroxyglutarate, galactosamine, glucose, and erythritol, capable of assessing a 5-year risk of pancreatic cancer has been discovered. Such methods are also useful in the treatment of patients with PDAC, permitting identification of PDAC patients with high sensitivity and specificity, at an early stage, and distinguished from patients with other prostate conditions such as benign prostate hyperplasia.

### SUMMARY

Provided herein is a method of treatment of pancreatic ductal adenocarcinoma (PDAC) in a patient having an elevated risk score or positive risk profile based on the patient's measured levels of trimethylamine N-oxide (TMAO), indoleacrylic acid, and an indole derivative derived from the metabolism of tryptophan by intestinal microorganisms, and optionally, levels of cholesterol glucuronide, 2-hydroxyglutarate, galactosamine, glucose, erythritol, and CA19-9, wherein the elevated risk score or positive risk profile led to the patient's diagnosis with PDAC, comprising administering a therapeutically effective amount of a treatment for PDAC to the patient.

Also provided herein is a method of treatment of pancreatic ductal adenocarcinoma (PDAC), comprising:
a) identifying a patient with an elevated risk score or positive risk profile based on the patient's measured levels of trimethylamine N-oxide (TMAO), indoleacrylic acid, and an indole derivative derived from the metabolism of tryptophan by intestinal microorganisms, and optionally, levels of cholesterol glucuronide, 2-hydroxyglutarate, galactosamine, glucose, erythritol, and CA19-9, wherein the elevated risk score or positive risk profile led to the patient's diagnosis with PDAC; and
b) administering a therapeutically effective amount of a treatment for PDAC to the patient.

Also provided herein is a method of determining the risk of a subject for pancreatic ductal adenocarcinoma (PDAC), comprising, in a biological sample obtained from the subject:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in the biological sample; and
b) classifying the subject as being at risk of PDAC or not at risk of PDAC based on the measured levels.

Also provided herein is a method of producing a risk profile of a subject for pancreatic ductal adenocarcinoma (PDAC), comprising, in a biological sample obtained from the subject:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in the biological sample; and
b) classifying the risk profile of the subject as being at risk of PDAC (positive) or not at risk of PDAC (negative) based on the measured levels.

Also provided herein is a method for calculating a patient's biomarker scores or risk score for PDAC, comprising:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in a biological sample obtained from the patient; and
b) calculating the biomarker scores or risk score using the numerical values of the measured levels in a machine learning model.

Also provided herein is a method of risk stratification for a patient at risk for PDAC, comprising, in a biological sample obtained from the patient:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in the biological sample; and
b) determining, by processor circuitry, the risk score for the patient, wherein the risk score is determined via a scoring function derived from metabolite profiles for biological samples taken from a plurality of individuals that were monitored for PDAC.

### BRIEF DESCRIPTION OF THE DRAWINGS

FIG. 1 depicts the workflow of analyses of the different datasets.

FIG. 2 depicts the relationship between TMAO and different microbial species. Data was derived from the Metabolomics Data Explorer database. Cs, Bt, and Pd are increased/elevated in fecal samples of PDAC patients compared to controls. Er is decreased in fecal samples of PDAC patients compared to controls. Ca abundance in fecal samples is associated with poor prognosis among PDAC patients. Et is associated with hepatobiliary disease, including pancreatic cancer.

FIG. 3 depicts the association between indoleacrylic acid and different microbial species. Data was derived from the Metabolomics Data Explorer database. Cs, Bt, and Pd are increased/elevated in fecal samples of PDAC patients compared to controls. Er is decreased in fecal samples of PDAC patients compared to controls. Ca abundance in fecal samples is associated with poor prognosis among PDAC patients. Et is associated with hepatobiliary disease, including pancreatic cancer.

FIG. 4 depicts the predictive performance of the 3MMP in the independent newly diagnosed PDAC cohort for resectable PDAC cases vs. healthy controls. CP: chronic pancreatitis.

FIG. 5 depicts the predictive performance of the 3MMP in the independent newly diagnosed PDAC cohort for individuals with CP (chronic pancreatitis) vs. healthy controls.

FIG. 6 depicts the predictive performance of the 3MMP in the independent newly diagnosed PDAC cohort for PDAC and CP (chronic pancreatitis) cases vs. healthy controls.

FIG. 7 depicts the absolute 5-year risk estimates for individuals with 3MMP panel scores. Vertical lines represent 20, 40, 60, and 80 percentiles values.

FIG. 8 depicts the absolute 5-year risk estimates for individuals with 3MMP+CA19-9 panel scores. Vertical lines represent 20, 40, 60, and 80 percentiles values.

FIG. 9 depicts odds ratios and adjusted odds ratios for individual microbiome-related metabolites for risk of pancreatic cancer in the Training Set. Sex, age, smoking status, and BMI were included as covariates in the adjusted odds ratios.

FIG. 10 depicts a Spearman correlation heatmap for microbiome-related metabolites in the Training Set.

FIG. 11 depicts the absolute 5-year risk estimates for individuals with 3MMP+5-marker non-microbial panel+CA19-9 panel scores. Vertical lines represent 20, 40, 60, and 80 percentiles values.

FIG. 12 depicts AUC estimates for the metabolite (host+microbial-derived) panel based on parametric empirical Bayes (PEB) or single-threshold (ST) methods.

FIG. 13 depicts a dot plot illustrating biomarker scores from the 3-marker microbial-derived panel in serially collected case plasmas. The X-axis represents the time (in years) from blood draw to the clinical diagnosis of PDAC among cases. Connecting lines between nodes depict serial samples from the same individual.

FIG. 14 depicts a dot plot showing the median (+/− standard error) of the 5-marker host-derived panel among cases (blue line) and non-case ‘controls’ (red line) when considering specimens collected within 5 years of diagnosis for cases or equivalent study follow-up time for non-cases. A fitted spline curve is included.

### DETAILED DESCRIPTION

Provided herein is a method of treatment of pancreatic ductal adenocarcinoma (PDAC) in a patient having an elevated risk score or positive risk profile based on the patient's measured levels of trimethylamine N-oxide (TMAO), indoleacrylic acid, and an indole derivative derived from the metabolism of tryptophan by intestinal microorganisms, and optionally, levels of cholesterol glucuronide, 2-hydroxyglutarate, galactosamine, glucose, erythritol, and CA19-9, wherein the elevated risk score or positive risk profile led to the patient's diagnosis with PDAC, comprising administering a therapeutically effective amount of a treatment for PDAC to the patient.

Also provided herein is a method of treatment of pancreatic ductal adenocarcinoma (PDAC), comprising:
a) identifying a patient with an elevated risk score or positive risk profile based on the patient's measured levels of trimethylamine N-oxide (TMAO), indoleacrylic acid, and an indole derivative derived from the metabolism of tryptophan by intestinal microorganisms, and optionally, levels of cholesterol glucuronide, 2-hydroxyglutarate, galactosamine, glucose, erythritol, and CA19-9, wherein the elevated risk score or positive risk profile led to the patient's diagnosis with PDAC; and
b) administering a therapeutically effective amount of a treatment for PDAC to the patient.

Also provided herein is a method of determining the risk of a subject for pancreatic ductal adenocarcinoma (PDAC), comprising, in a biological sample obtained from the subject:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in the biological sample; and
b) classifying the subject as being at risk of PDAC or not at risk of PDAC based on the measured levels.

Also provided herein is a method of producing a risk profile of a subject for pancreatic ductal adenocarcinoma (PDAC), comprising, in a biological sample obtained from the subject:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in the biological sample; and
b) classifying the risk profile of the subject as being at risk of PDAC (positive) or not at risk of PDAC (negative) based on the measured levels.

Also provided herein is a method for calculating a patient's biomarker scores or risk score for PDAC, comprising:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in a biological sample obtained from the patient; and
b) calculating the biomarker scores or risk score using the numerical values of the measured levels in a machine learning model.

Also provided herein is a method of risk stratification for a patient at risk for PDAC, comprising, in a biological sample obtained from the patient:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in the biological sample; and
b) determining, by processor circuitry, the risk score for the patient, wherein the risk score is determined via a scoring function derived from metabolite profiles for biological samples taken from a plurality of individuals that were monitored for PDAC.

In some embodiments, the machine learning model is a logistic regression.

In some embodiments, the machine learning model is a LASSO regularization.

In some embodiments, the biomarker scores or risk score for PDAC are/is calculated with the equation: 0.3653*[indoleacrylic acid]+0.2412*[TMAO]+0.5022*[indole derivative].

In some embodiments, the biomarker scores or risk score for PDAC are/is calculated with the equation: 1.478*[alpha-D-glucose]+14.941*[cholesterol glucuronide]+5.415*[galactosamine]+4.206*[D-2-hydroxyglutarate]+−1.653*[erythritol]

In some embodiments, the biomarker scores or risk score for PDAC are/is calculated with the equation: 0.023*[CA19-9]+1.425*(0.3653*[indoleacrylic acid]+0.2412*[TMAO]+0.5022*[indole derivative])+0.872*(1.478*[alpha-D-glucose]+14.941*[cholesterol glucuronide]+5.415*[galactosamine]+4.206*[D-2-hydroxyglutarate]+—1.653*[erythritol]).

In some embodiments, the method further comprises measuring the levels of or identifying a patient with elevated levels of cholesterol glucuronide, 2-hydroxyglutarate, galactosamine, glucose, and erythritol.

In some embodiments, the method further comprises measuring the level of CA19-9.

In some embodiments, the indole derivative has a molecular weight of less than 500 daltons.

In some embodiments, the indole derivative of is a compound of structural Formula I:

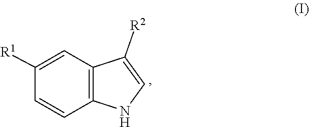

wherein:
R¹ is chosen from hydrogen, hydroxy, and C₁₋₄alkoxy, any of which may be optionally substituted;
R² is chosen from hydrogen,

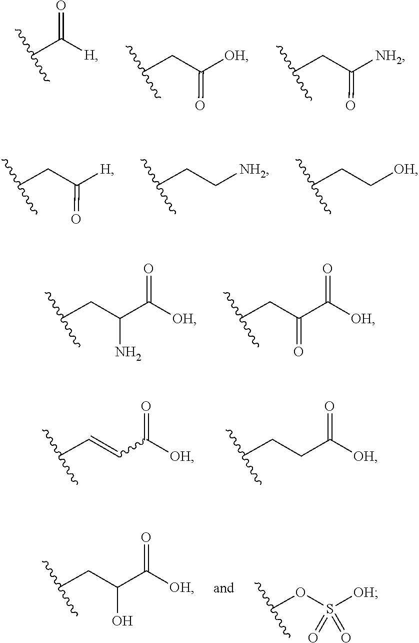

and
the compound of Formula I is not chosen from 5-hydroxy-L-tryptophan, 5-methoxy-3-indoleacetic acid, indole-3-lactic acid, indole-3-acetaldehyde, indole-3-ethanol, indole-3-acetamide, and indole-3-acetate.

In some embodiments, the indole derivative has a molecular weight of about 177 daltons.

In some embodiments, the risk score based on the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, is elevated relative to a reference patient or group that does not have PDAC.

In some embodiments, the risk score based on the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, is elevated in comparison to the levels in a reference patient or group that has chronic pancreatitis or benign pancreatic disease.

In some embodiments, the PDAC is diagnosed at or before the borderline resectable stage or at the resectable stage.

In some embodiments, the patient is over 50 years old.

In some embodiments, the patient has new-onset diabetes mellitus, or an asymptomatic variant thereof.

In some embodiments, the patient has chronic pancreatitis, or an asymptomatic variant thereof.

In some embodiments, the patient has been incidentally diagnosed with mucin-secreting cysts of the pancreas, or an asymptomatic variant thereof.

In some embodiments, each of trimethylamine N-oxide (TMAO), indoleacrylic acid, and an indole derivative derived from the metabolism of tryptophan by intestinal microorganisms, and optionally, each of cholesterol glucuronide, 2-hydroxyglutarate, galactosamine, glucose, erythritol, and CA19-9, generates a detectable signal.

In some embodiments, the detectable signals are detectable by a spectrometric method.

In some embodiments, the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

In some embodiments, the spectrometric method is mass spectrometry.

In some embodiments, the mass spectrometry is LC-TOF-MS.

In some embodiments, the treatment is chosen from surgery, chemotherapy, immunotherapy, radiation therapy, targeted therapy, or a combination thereof.

In some embodiments, the calculated biomarker scores, risk score, or risk profile are/is based on sensitivity and specificity values that corresponds to the risk threshold of the subject for PDAC.

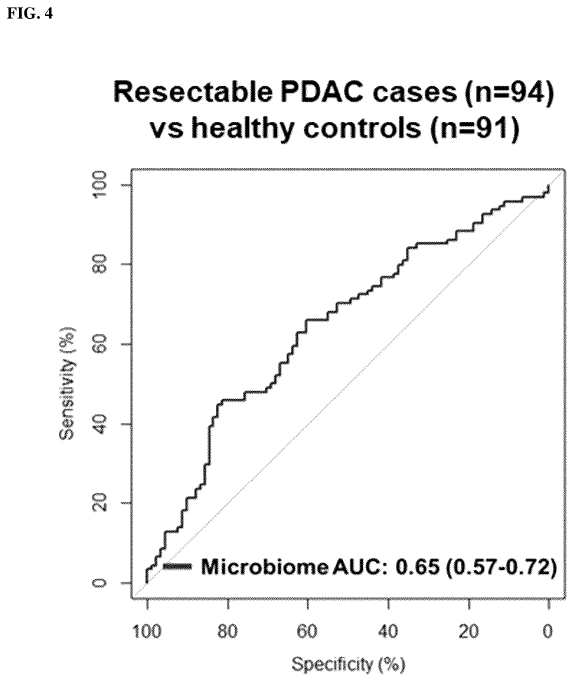

In some embodiments, the risk profile has sensitivity and specificity values that do not differ substantially from the curve in FIG. 4.

In some embodiments, the sensitivity and specificity values differ by less than 10%.

In some embodiments, the sensitivity and specificity values differ by less than 5%.

In some embodiments, the sensitivity and specificity values differ by less than 1%.

In some embodiments, the cutoff point comprises an AUC (95% CI) of at least 0.57.

In some embodiments, the AUC of the method is greater than 0.77.

In some embodiments, the AUC of the method is between 0.77 and 0.95.

In some embodiments, the AUC of the method is about 0.86.

In some embodiments, the AUC of the method is 0.86.

In some embodiments, the AUC of the method is about 0.84.

In some embodiments, the AUC of the method is 0.84.

In some embodiments, the AUC of the method is about 0.79.

In some embodiments, the AUC of the method is 0.79.

In some embodiments, the AUC of the method is greater than the AUC for a different biomarker, biomarkers, panel, assay, algorithm, model, or any combination thereof.

In some embodiments, the biomarker is CA19-9 alone.

In some embodiments, the cutoff points of the respective AUCs are used for comparison.

In some embodiments, the methods are analyzed by the same statistical methods.

In some embodiments, the 5-year odds ratio (OR) for the probability of developing PDAC is between 1.13 and 2.23.

In some embodiments, the OR is between 1.3 and 1.8.

In some embodiments, the OR is about 1.55.

In some embodiments, the OR is 1.55.

In some embodiments, the method further comprises assigning the patient to an appropriate risk group based on the calculated risk score.

In some embodiments, there are at least two risk groups.

In some embodiments, the adjusted odds ratio (AOR) for the probability of developing PDAC per unit standard deviation (SD) is between 0.98 and 47.66.

In some embodiments, the AOR is between 1.5 and 10.0.

In some embodiments, the AOR is about 1.72.

In some embodiments, the AOR is 1.72.

In some embodiments, the AOR is about 3.13.

In some embodiments, the AOR is 3.13.

In some embodiments, the AOR is about 9.67.

In some embodiments, the AOR is 9.67.

In some embodiments, the AOR represents the odds of developing PDAC within the next 5 years.

In some embodiments, the AOR is between 1.4 and 15.0.

In some embodiments, the AOR is about 1.43.

In some embodiments, the AOR is 1.43.

In some embodiments, the AOR is about 3.8.

In some embodiments, the AOR is 3.8.

In some embodiments, the AOR is about 14.99.

In some embodiments, the AOR is 14.99.

In some embodiments, the AOR represents the odds of developing PDAC within the next 2 years.

In some embodiments, the AOR is between 1.5 and 6.0.

In some embodiments, the AOR is about 2.11.

In some embodiments, the AOR is 2.11.

In some embodiments, the AOR is about 1.90.

In some embodiments, the AOR is 1.90.

In some embodiments, the AOR is about 5.10.

In some embodiments, the AOR is 5.10.

In some embodiments, the AOR represents the odds of developing PDAC within the next 2 to 5 years.

In some embodiments, the AOR value is controlled for age, sex, smoking and body mass index (BMI).

In some embodiments, the AOR value is additionally controlled for diabetic status.

In some embodiments, the risk score is measured against a given threshold value that represents the absolute risk of developing PDAC over the next five years.

In some embodiments, the threshold value is greater than 0.001, or 0.1%.

In some embodiments, the threshold value is between 0.005 and 0.1, or 0.5% and 10%.

In some embodiments, the threshold value is about 0.01, or 1%.

In some embodiments, the threshold value is 0.01, or 1%.

In some embodiments, the risk score exceeds the threshold value and the patient is classified as being at risk for PDAC.

In some embodiments, the risk score is below the threshold value and the patient is classified as being not at risk for PDAC.

In some embodiments, the patient is subsequently designated for pancreatic cancer screening.

In some embodiments, the screening is chosen from endoscopic ultrasound, magnetic resonance imaging (MRI), and computed topography (CT) scans.

In some embodiments, the screening is performed annually.

In some embodiments, the screening is performed semi-annually.

In some embodiments, the method further comprises measuring the levels of trimethylamine N-oxide (TMAO), indoleacrylic acid, and/or an indole derivative derived from the metabolism of tryptophan by intestinal microorganisms, and optionally, levels of cholesterol glucuronide, 2-hydroxyglutarate, galactosamine, glucose, erythritol, and CA19-9, in at least one or more additional biological samples obtained from the patient and classifying the patient with an elevated risk score or positive risk profile OR being at risk or not at risk of PDAC.

In some embodiments, the patient classification is calculated in a machine learning model.

In some embodiments, the machine learning model is a parametric empirical Bayes longitudinal algorithm.

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “hydroxy,” as used herein, alone or in combination, refers to —OH.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g., aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

As used herein, the term “pancreatic cancer” means a malignant neoplasm of the pancreas characterized by the abnormal proliferation of cells, the growth of which cells exceeds and is uncoordinated with that of the normal tissues around it.

As used herein, the term “PDAC” refers to pancreatic ductal adenocarcinoma, which is pancreatic cancer that can originate in the ducts of the pancreas.

As used herein, the term “pancreatitis” refers to an inflammation of the pancreas. Pancreatitis is not generally classified as a cancer, although it may advance to pancreatic cancer.

As used herein, the term “subject” or “patient” as used herein refers to a mammal, preferably a human, for whom a classification as PDAC-positive or PDAC-negative is desired, and for whom further treatment can be provided.

As used herein, a “reference patient” or “reference group” refers to a group of patients or subjects to which a test sample from a patient suspected of having or being susceptible to PDAC may be compared. In some embodiments, such a comparison may be used to determine whether the test subject has PDAC. A reference patient or group may serve as a control for testing or diagnostic purposes. As described herein, a reference patient or group may be a sample obtained from a single patient, or may represent a group of samples, such as a pooled group of samples.

As used herein, “healthy” refers to an individual having a healthy pancreas, or normal, non-compromised pancreatic function. A healthy patient or subject has no symptoms of PDAC or other pancreatic disease. In some embodiments, a healthy patient or subject may be used as a reference patient for comparison to diseased or suspected diseased samples for determination of PDAC in a patient or a group of patients.

As used herein, “treating,” “treatment,” and the like means the administration of therapy to an individual who already manifests at least one symptom of a disease or condition or who has previously manifested at least one symptom of a disease or condition. For example, “treating” can include alleviating, abating, or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder. As related to the present disclosure, the term may also mean the administration of pharmacological substances or formulations, or the performance of non-pharmacological methods including, but not limited to, radiation therapy and surgery. Pharmacological substances as used herein may include, but are not limited to, chemotherapeutics that are established in the art, such as Gemcitabine (GEMZAR), 5-fluorouracil (5-FU), irinotecan (CAMPTOSAR), oxaliplatin (ELOXATIN), albumin-bound paclitaxel (ABRAXANE), capecitabine (XELODA), cisplatin, paclitaxel (TAXOL), docetaxel (TAXOTERE), and irinotecan liposome (ONIVYDE). Pharmacological substances may include substances used in immunotherapy, such as checkpoint inhibitors. Treatment may include a multiplicity of pharmacological substances, or a multiplicity of treatment methods, including, but not limited to, surgery and chemotherapy.

As used herein, the term “ELISA” refers to enzyme-linked immunosorbent assay. This assay generally involves contacting a fluorescently tagged sample of proteins with antibodies having specific affinity for those proteins. Detection of these proteins can be accomplished with a variety of means, including but not limited to laser fluorimetry.

As used herein, the term “regression” refers to a statistical method that can assign a predictive value for an underlying characteristic of a sample based on an observable trait (or set of observable traits) of said sample. In some embodiments, the characteristic is not directly observable. For example, the regression methods used herein can link a qualitative or quantitative outcome of a particular biomarker test, or set of biomarker tests, on a certain subject, to a probability that said subject is for PDAC-positive.

As used herein, the term “logistic regression” refers to a regression method in which the assignment of a prediction from the model can have one of several allowed discrete values. For example, the logistic regression models used herein can assign a prediction, for a certain subject, of either PDAC-positive or PDAC-negative.

As used herein, “amount” or “level” refers to a typically quantifiable measurement for a biomarker described herein, wherein the measurement enables comparison of the marker between samples and/or to control samples. In some embodiments, an amount or level is quantifiable and refers to the levels of a particular marker in a biological sample (e.g., blood, serum, urine, etc.), as determined by laboratory methods or tests such as an immunoassay, (e.g., antibodies), mass spectrometry, or liquid chromatography. In some embodiments, a marker may be present in the sample in an increased amount, or in a decreased amount. Marker comparisons may be based on direct measurement of the levels of a biomarker described herein, (e.g., through protein quantification or gene expression analysis) or may be based on measurement of e.g., reporter molecules, biomarker-receptor complexes, biomarker-relay-receptor complexes, or the like.

As used herein, the term “elevated” refers to a biomarker level or risk score in a given subject that is greater relative to the same biomarker level or model score in a given set of healthy patients or subjects.

As used herein, the term “biomarker score” refers to a numerical score for a given biomarker measured in a sample from a subject. The biomarker score is calculated by normalizing or weighting the measured level using a fixed coefficient as prescribed by the statistical method for a given biomarker panel. Biomarker scores are used as components in calculating a risk score for the subject. Elevated biomarker scores will carry more weight in risk score calculations and can indicate a higher risk for PDAC for the subject.

As used herein, the term “risk score” refers to a single numerical value that indicates an asymptomatic human subject's risk for PDAC as compared to the known prevalence in the disease cohort. The risk score is calculated through adding together the parameters of a statistical method derived from the subject for a given biomarker panel, which may take the form of biomarker scores, statistical model scores, or model constants. A higher risk score correlates to a higher risk for PDAC in the subject. The risk score is empirically derived and will change depending on the data, cohort of the subject population, type of cancer, biomarkers chosen, occupational and environmental factors, and so on. In certain embodiments, the risk score as calculated for the human subject is the summation of the biomarker scores obtained from the subject. In certain embodiments, the risk score as calculated for the human subject is the summation of the biomarker scores obtained from the subject and one or more additional model constants. In certain embodiments, the risk score as calculated for a human subject is the summation of the biomarker scores obtained for the subject, normalized scores from one or more additional statistical models based on risk factors for the subject, and one or more additional model constants.

As used herein, the term “risk profile” refers to an assessment of a patient's risk score compared to those of a plurality of patients assessed using the same model, in which the patient is placed into an appropriate risk group based on a given score threshold. The score threshold is empirically derived and will change depending on the data, cohort of the subject population, type of cancer, biomarkers chosen, occupational and environmental factors, and so on. In certain embodiments, the patient's risk score exceeds the score threshold and their risk profile classifies them as being at risk for PDAC (“positive”). In certain embodiments, the patient's risk profile is lower than the score threshold and classifies them as not being at risk for PDAC (“negative”). In some embodiments, the score threshold is 0.001, or 0.1%, or greater. In some embodiments, the score threshold is 0.005, or 0.5%, or greater. In some embodiments, the score threshold is 0.01, or 1%, or greater. In some embodiments, the score threshold is 0.05, or 5%, or greater. In some embodiments, the score threshold is 0.1, or 10%, or greater.

As used herein, the term “cutoff point” refers to a mathematical value associated with a specific statistical method that can be used to assign a classification of PDAC-positive of PDAC-negative to a subject, based on said subject's biomarker score.

As used herein, when a numerical value above or below a cutoff value “is characteristic of PDAC,” what is meant is that the subject, analysis of whose sample yielded the value, either has PDAC or is at risk for PDAC.

As used herein, the term “classification” refers to the assignment of a subject as either PDAC-positive or PDAC-negative, based on the result of the risk score or biomarker scores that is/are obtained for said subject.

As used herein, the term “PDAC-positive” refers to an indication that a subject is predicted as susceptible to PDAC, based on the results of the outcome of the methods of the disclosure.

As used herein, the term “PDAC-negative” refers to an indication that a subject is predicted as not susceptible to PDAC, based on the results of the outcome of the methods of the disclosure.

As used herein, the term “Wilcoxon rank sum test,” also known as the Mann-Whitney U test, Mann-Whitney-Wilcoxon test, or Wilcoxon-Mann-Whitney test, refers to a specific statistical method used for comparison of two populations. For example, the test can be used herein to link an observable trait, in particular a biomarker level, to the absence or presence of PDAC in subjects of a certain population.

As used herein, the term “ROC” refers to receiver operating characteristic, which is a graphical plot used herein to gauge the performance of a certain diagnostic method at various cutoff points. A ROC plot can be constructed from the fraction of true positives and false positives at various cutoff points.

As used herein, the term “AUC” refers to the area under the curve of the ROC plot. AUC can be used to estimate the predictive power of a certain diagnostic test. Generally, a larger AUC corresponds to increasing predictive power, with decreasing frequency of prediction errors. Possible values of AUC range from 0.5 to 1.0, with the latter value being characteristic of an error-free prediction method.

As used herein, the term “p-value” or “p” refers to the probability that the distributions of biomarker scores for positive-PDAC and non-positive-PDAC subjects are identical in the context of a Wilcoxon rank sum test. Generally, ap-value close to zero indicates that a particular statistical method will have high predictive power in classifying a subject.

As used herein, the term “CI” refers to a confidence interval, i.e., an interval in which a certain value can be predicted to lie with a certain level of confidence. As used herein, the term “95% CI” refers to an interval in which a certain value can be predicted to lie with a 95% level of confidence.

As used herein, the term “3-marker microbial-related metabolite panel” or “3MMP” refers to a panel of three biomarkers, which includes TMAO, indoleacrylic acid, and an indole derivative, useful for assessing the risk of PDAC in a patient suspected of being at risk for PDAC. In some embodiments, the 3MMP may be evaluated in combination with additional biomarkers or statistical models to enhance the prediction of PDAC in biological samples from patients suspected as being at risk for PDAC. Useful biomarkers include, but are not limited to, cholesterol glucuronide, 2-hydroxyglutarate, galactosamine, glucose, erythritol, and CA19-9.

As used herein, an “indole-derivative” refers to compounds that are derived from indole. Indole is an aromatic heterocyclic organic compound with formula C₈H₇N. It has a bicyclic structure, consisting of a six-membered benzene ring fused to a five-membered nitrogen-containing pyrrole ring. An indole-derivative as described herein may be any derivative of indole.

In some embodiments, using markers TMAO, indoleacrylic acid, and an indole derivative together as a panel, or using markers TMAO, indoleacrylic acid, an indole derivative, cholesterol glucuronide, 2-hydroxyglutarate, galactosamine, glucose, erythritol, and CA19-9 together as a panel may have an AUC (95% CI) of 0.57 or greater, including about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

As used herein, the term “sensitivity” refers to, in the context of various biochemical assays, the ability of an assay to correctly identify those with a disease (i.e., the true positive rate). By comparison, as used herein, the term “specificity” refers to, in the context of various biochemical assays, the ability of an assay to correctly identify those without the disease (i.e., the true negative rate). Sensitivity and specificity are statistical measures of the performance of a binary classification test (i.e., classification function). Sensitivity quantifies the avoiding of false negatives, and specificity does the same for false positives.

As used herein, “fixed coefficients” or “fixed model coefficients” refers to a statistical method of standardizing coefficients in order to allow comparison of the relative importance of each coefficient in a regression model. In some embodiments, fixed coefficients involve using the same beta-coefficients from a logistic regression model to yield a risk score for the developed combination rule, which is ultimately used to make a clinical decision based on a decision threshold(s).

As used herein, a “sample” refers to a test substance to be tested for the presence of, and levels or concentrations thereof, of a biomarker as described herein. A sample may be any substance appropriate in accordance with the present disclosure, including, but not limited to, blood, blood serum, blood plasma, or any part thereof.

As used herein, the term “CA19-9” refers to carbohydrate antigen 19-9, and is also known in the art as cancer antigen 19-9 and sialylated Lewis antigen.

As used herein, a “metabolite” refers to small molecules that are intermediates and/or products of cellular metabolism. Metabolites may perform a variety of functions in a cell, for example, structural, signaling, stimulatory and/or inhibitory effects on enzymes. In some embodiments, a metabolite may be a non-protein, plasma-derived metabolite marker, such as including, but not limited to, acetylspermidine, diacetylspermine, lysophosphatidylcholine (18:0), lysophosphatidylcholine (20:3), TMAO, indoleacrylic acid, an indole derivative, cholesterol glucuronide, 2-hydroxyglutarate, galactosamine, glucose, and erythritol.

The phrase “therapeutically effective” is intended to qualify the amount of active ingredients used in the treatment of a disease or disorder or on the effecting of a clinical endpoint.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

### Diagnosis, Staging, and Treatment of Pancreatic Cancer.

The most common way to classify pancreatic cancer is to divide it into 4 categories based on whether it can be removed with surgery and where it has spread: resectable, borderline resectable, locally advanced, or metastatic. Resectable pancreatic cancer can be surgically removed. The tumor may be located only in the pancreas or extends beyond it, but it has not grown into important arteries or veins in the area. There is no evidence that the tumor has spread to areas outside of the pancreas. Using standard methods common in the medical industry today, only about 10% to 15% of patients are diagnosed with this stage. Borderline resectable describes a tumor that may be difficult, or not possible, to remove surgically when it is first diagnosed, but if chemotherapy and/or radiation therapy is able to shrink the tumor first, it may be able to be removed later with negative margins. A negative margin means that no visible cancer cells are left behind. Locally advanced pancreatic cancer is still located only in the area around the pancreas, but it cannot be surgically removed because it has grown into nearby arteries or veins or to nearby organs. However, there are no signs that it has spread to any distant parts of the body. Using standard methods common in the medical industry today, approximately 35% to 40% of patients are diagnosed with this stage. Metastatic means the cancer has spread beyond the area of the pancreas and to other organs, such as the liver or distant areas of the abdomen. Using standard methods common in the medical industry today, approximately 45% to 55% of patients are diagnosed with this stage. Alternatively, the TNM Staging System, commonly used for other cancers, may be used (but is not common in pancreatic cancer). This system is based on tumor size (T), spread to lymph nodes (N), and metastasis (M).

Options for treatment of pancreatic cancer include surgery for partial or complete surgical removal of cancerous tissue (for example a Whipple procedure, distal pancreatectomy, or total pancreatectomy), administering one or more chemotherapeutic drugs, and administering therapeutic radiation to the affected tissue (e.g., conventional/standard fraction radiation therapy stereotactic body radiation (SBRT)). Chemotherapeutic drugs approved for treatment of pancreatic cancer include, but are not limited to, capecitabine (Xeloda), erlotinib (Tarceva), fluorouracil (5-FU), gemcitabine (Gemzar), irinotecan (Camptosar), leucovorin (Wellcovorin), nab-paclitaxel (Abraxane), nanoliposomal irinotecan (Onivyde), and oxaliplatin (Eloxatin).

Pancreatic cancer is treated most effectively when diagnosed early, preferably at or before the borderline resectable stage and more preferably at the resectable stage.

### EXAMPLES

The invention is further illustrated by the following examples.

### Example 1: Test Sets

### PLCO Cohort

The PLCO Cancer Screening Trial is a randomized multicenter trial in the United States which aims to evaluate the impact of early detection procedures for prostate, lung, colorectal and ovarian cancer on disease-specific mortality. All subjects involved in this study were enrolled with written consent as a criterion for eligibility to participate in the PLCO trial. Study recruitment and randomization began November 1993 and was completed in July 2001. PLCO eligibility criteria excluded subjects with a previous personal history of PLCO cancers, ongoing cancer treatment (excluding basal-cell and squamous-cell skin cancer), participation in another cancer screening or cancer primary prevention trial, and a recent screening test for prostate or colorectal cancer. The cohort comprises approximately 155,000 men and women aged 55 to 74 years old at baseline entry. Study participants completed a baseline questionnaire at study entry that includes demographic, personal, and medical information including diabetes status.

Sera from 175 pancreatic cancer cases that were diagnosed within 5 years of blood draw and 875 matched non-cases from 10 participating PLCO study centers (Table 1) were analyzed. Pancreatic cancer cases were identified by self-report in annual mail-in surveys, state cancer registries, death certificates, physician referrals and reports from next of kin for deceased individuals. All medical and pathologic records related to pancreatic cancer diagnosis and supporting documentation were obtained and confirmed by PLCO staff. Pancreatic cancers were classified as localized, regional, distant, or unstaged using the National Cancer Institute Surveillance, Epidemiology, and End Results (SEER) historic staging system. Non-cases, alive at the time when the index case was diagnosed, were matched to cases at a ratio of 5:1 (non-case:case) based on the distribution of age, race, sex, and calendar date of blood draw in 2-month blocks within the case cohort.

TABLE 1
Patient and Tumor
characteristics for PLCO cohort

| | Case/Control Status | | | |
|---|---|---|---|---|
| | Non-Case | | Case | |
| | N | % | N | % |
| Total | 875 | 100 | 175 | 100 |

Gender

| | | | | |
|---|---|---|---|---|
| Female | 365 | 41.71 | 73 | 41.71 |
| Male | 510 | 58.29 | 102 | 58.29 |

Age At Randomization

| | | | | |
|---|---|---|---|---|
| <=59 | 185 | 21.14 | 37 | 21.14 |
| 60-64 | 210 | 24 | 42 | 24 |
| 65-69 | 325 | 37.14 | 65 | 37.14 |
| >=70 | 155 | 17.71 | 31 | 17.71 |

Race

| | | | | |
|---|---|---|---|---|
| White | 795 | 90.86 | 159 | 90.86 |
| Black | 30 | 3.43 | 6 | 3.43 |
| Other | 50 | 5.71 | 10 | 5.71 |

Cigarette Smoking Status

| | | | | |
|---|---|---|---|---|
| Never Smoked | 426 | 48.69 | 65 | 37.14 |
| Cigarettes | | | | |
| Current Cigarette | 74 | 8.46 | 36 | 20.57 |
| Smoker | | | | |
| Former Cigarette | 375 | 42.86 | 74 | 42.29 |
| Smoker | | | | |

BMI at Baseline (In kg/m2)

| | | | | |
|---|---|---|---|---|
| Not Answered | 7 | 0.8 | 0 | 0 |
| 0-18.5 | 8 | 0.91 | 3 | 1.71 |
| 18.5-25 | 306 | 34.97 | 57 | 32.57 |
| 25-30 | 368 | 42.06 | 72 | 41.14 |
| 30+ | 186 | 21.26 | 43 | 24.57 |

Diabetic Status

| | | | | |
|---|---|---|---|---|
| Unknown | 1 | 0.11 | — | — |
| Yes | 55 | 6.292 | 23 | 13.14 |
| No | 819 | 96.6 | 152 | 86.86 |

SEER Staging (cases only)

| | | |
|---|---|---|
| Unknown | 9 | 5.14 |
| Localized | 35 | 20 |
| Regional | 33 | 18.86 |
| Distant | 92 | 52.57 |

Newly Diagnosed Pancreatic Cancer Cohort

An independent test set consisted of plasma samples from 99 patients with resected PDAC, 50 patients with chronic pancreatitis, and 100 healthy controls as previously described (Table 2). Patients with pancreatic cancer provided informed written consent to collection of pretreatment plasma samples and clinical data abstraction. Patients with PDAC were recruited from cancer clinics at Dana-Farber Cancer Institute/Brigham and Women's Hospital (DFCBWH), Beth Israel Deaconess Medical Center (BIDMC), and Columbia University Irving Medical Center (CUIMC). Healthy control patients were recruited from DFCUBWH and CUIMC. Healthy controls were undergoing screening colonoscopy or accompanying a non-blood-related patient to an appointment at a gastrointestinal cancer clinic. Healthy controls had no history of cancer in the 5 years before sample collection. Patients with pancreatic cancer and healthy controls were matched on gender and age at the time of blood collection. Patients with chronic pancreatitis (CP) were recruited from gastroenterology clinics at DFCUIBWH, BIDMC, and CUIMC. Patients were included if clinic notes from a gastroenterologist indicated a diagnosis of CP. Patients with pancreatic cancer or CP were not gender or age matched. Clinical data abstraction was performed identically across the sites with data uploaded to a password-protected REDCap database. All plasma samples were collected and processed according to a uniform, standardized protocol across the sites and patient groups.

TABLE 2
Patient and tumor characteristics for
the newly diagnosed PDAC cohort.

| | | | Chronic | | Healthy | |
|---|---|---|---|---|---|---|
| | PDAC Case | | Pancreatitis | | Control | |
| | (N = 99) | | (N = 50) | | (N = 100) | |
| Variable | No. | % | No. | % | No. | % |
| Institution | | | | | | |
| DF/BWCC | 69 | 70% | 30 | 60% | 94 | 94% |
| BIDMC | 15 | 15% | 15 | 30% | 0 | 0% |
| CUMC | 15 | 15% | 5 | 10% | 6 | 6% |
| Age (year), median | 69.8 (62.5- | | 65.4 (54.7- | | 63.7 (55.7- | |
| (IQR) | 74.8) | | 72.2) | | 70.6) | |
| Gender | | | | | | |
| Male | 51 | 52% | 33 | 66% | 51 | 51% |
| Female | 48 | 48% | 17 | 34% | 49 | 49% |
| Race | | | | | | |
| White | 94 | 95% | 42 | 84% | 84 | 86% |
| Black/African-American | 0 | 0% | 5 | 10% | 5 | 5% |
| Asian | 1 | 1% | 0 | 0% | 2 | 2% |
| Other | 4 | 4% | 3 | 6% | 7 | 7% |
| Blood collection year | | | | | | |
| 2015-2016 | 19 | 19% | 2 | 4% | 0 | 0% |
| 2017-2019 | 80 | 81% | 48 | 96% | 100 | 100% |
| Smoking Status | | | | | | |
| Current Smoker | 6 | 6% | 11 | 22% | 4 | 4% |
| Past smoker | 50 | 51% | 17 | 34% | 42 | 42% |
| Never smoker | 43 | 43% | 22 | 44% | 54 | 54% |
| BMI (kg/m²), | 27.4 (24.0- | | 25.0 (22.8- | | 27.5 (24.3- | |
| Median(IQR) | 30.0) | | 27.6) | | 32.0) | |
| Diabetes | | | | | | |
| No | 64 | 65% | 23 | 46% | 93 | 93% |
| Yes | 35 | 35% | 27 | 54% | 7 | 7% |
| Etiology of chronic | | | | | | |
| pancreatitis | | | | | | |
| Alcohol | — | — | 16 | 32% | — | — |
| Autoimmune | — | — | 2 | 4% | — | — |
| Congenital anatomical | — | — | 3 | 6% | — | — |
| variant | | | | | | |
| Duct stricture or stones | — | — | 7 | 14% | — | — |
| Idiopathic | — | — | 21 | 42% | — | — |
| Other | — | — | 1 | 2% | — | — |
| AJCC 8^{th} edition | | | | | | |
| staging pTNM^{a} | | | | | | |
| T0-2N0M0 | 15 | 24% | — | — | — | — |
| T3-4N0M0 | 2 | 3% | — | — | — | — |
| T1-4N1M0 | 28 | 45% | — | — | — | — |
| T1-4N2M0 | 17 | 28% | — | — | — | — |
| AJCC 8^{th} edition | | | | | | |
| staging ypTNM^{b} | | | | | | |
| T0-2N0M0 | 24 | 64% | — | — | — | — |
| T3-4N0M0 | 1 | 3% | — | — | — | — |
| T1-4N1M0 | 7 | 19% | — | — | — | — |
| T1-4N2M0 | 5 | 14% | — | — | — | — |
| PDAC recurrence | | | | | | |
| No^{c} | 56 | 57% | — | — | — | — |
| Yes | 43 | 43% | — | — | — | — |

DF/BWCC: Dana-Farber/Brigham and Women's Cancer Center;
BIDMC: Beth Israel Deaconess Medical Center;
CUMC: Columbia University Medical Center
AJCC: American Joint Committee on Cancer,
PDAC: Pancreatic ductal adenocarcinoma,
BMI: Body mass index
^{a}Patients who underwent up-front surgical resection
^{b}Patients who received neoadjuvant treatment and then underwent surgical resection
^{c}The median (IQR) follow-up time was 15.0 (7.2-23.2) months for patients without cancer recurrence

### Example 2: Metabolomic Analysis

### Sample Extraction

Serum and plasma metabolites were extracted from pre-aliquoted biospecimen (15 μL) with 45 μL of LCMS grade methanol (ThermoFisher®) in a 96-well microplate (Eppendorf®). Plates were heat sealed, vortexed for 5 min at 750 rpm, and centrifuged at 2000×g for 10 minutes at room temperature. The supernatant (30 μL) was carefully transferred to a 96-well plate, leaving behind the precipitated protein. The supernatant was further diluted with 60 μL of 100 mM ammonium formate, pH 3 (Fisher Scientific®). For Hydrophilic Interaction Liquid Chromatography (HILIC) positive ion analysis, 15 μL of the supernatant and ammonium formate mix were diluted with 195 μL of 1:3:8:144 water (GenPure ultrapure water system, ThermoFisher®): LCMS grade methanol (ThermoFisher®): 100 mM ammonium formate, pH 3 (Fisher Scientific): LCMS grade acetonitrile (ThermoFisher®). For C18 analysis, 15 μL of the supernatant and ammonium formate mix were diluted with 90 μL water (GenPure ultrapure water system, ThermoFisher®) for positive ion mode. Each sample solution was transferred to 384-well microplate (Eppendorf®) for LCMS analysis.

### Untargeted Metabolomic Analyses

Untargeted metabolomics analyses were conducted on a Waters Acquity™ UPLC system with 2D column regeneration configuration (I-class and H-class) coupled to a Xevo G2-XS quadrupole time-of-flight (qTOF) mass spectrometer. Chromatographic separation was performed using HILIC (Acquity™ UPLC BEH amide, 100 Å, 1.7 μm 2.1×100 mm, Waters Corporation, Milford, U.S.A) and C18 (Acquity™ UPLC HSS T3, 100 Å, 1.8 μm, 2.1×100 mm, Water Corporation, Milford, U.S.A) columns at 45° C.

The quaternary solvent system mobile phases were (A) 0.1% formic acid in water, (B) 0.1% formic acid in acetonitrile and (D) 100 mM ammonium formate, pH 3. Samples were separated on the HILIC using the following gradient profile at 0.4 mL/min flow rate: (95% B, 5% D) linear change to (70% A, 25% B and 5% D) over 5 min; 100% A for 1 min; and 100% A for 1 min. For C18 separation, the chromatography gradient was as follows at 0.4 mL/min flow rate: 100% A with a linear change to (5% A, 95% B) over 5 min; (95% B, 5% D) for 1 min; and 1 min at (95% B, 5% D).

A binary pump was used for column regeneration and equilibration. The solvent system mobile phases were (A1) 100 mM ammonium formate, pH 3, (A2) 0.1% formic in 2-propanol and (B1) 0.1% formic acid in acetonitrile. The HILIC column was stripped using 90% A2 for 5 min at 0.25 mL/min flow rate, followed by a 2 min equilibration using 100% B1 at 0.3 mL/min flow rate. Reverse phase C18 column regeneration was performed using 95% A1, 5% B1 for 2 min followed by column equilibration using 5% A1, 95% B1 for 5 min at 0.4 mL/min flow rate.

### Mass Spectrometry Data Acquisition

Mass spectrometry data was acquired using ‘sensitivity’ mode in positive electrospray ionization mode within 50-800 Da range. For the electrospray acquisition, the capillary voltage was set at 1.5 kV (positive), sample cone voltage 30V, source temperature at 120° C., cone gas flow 50 L/h and desolvation gas flow rate of 800 L/h with scan time of 0.5 sec in continuum mode. Leucine Enkephalin; 556.2771 Da (positive) was used for lockspray correction and scans were performed at 0.5 sec. The injection volume for each sample was 6 μL. The acquisition was carried out with instrument auto gain control to optimize instrument sensitivity over the samples acquisition time.

### Data Processing

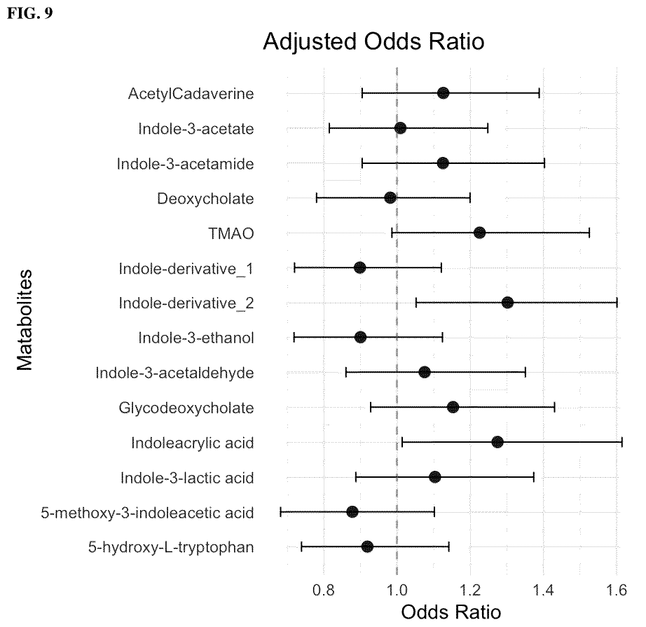

LC-MS and LC-MSe data were processed using Progenesis QI (Nonlinear, Waters). Peak picking and retention time alignment of LC-MS and MSe data were performed using Progenesis QI software (Nonlinear, Waters). Data processing and peak annotations were performed using an in-house automated pipeline. Annotations were determined by matching accurate mass and retention times using customized libraries created from authentic standards and by matching experimental tandem mass spectrometry data against the NIST MSMS, LipidBlast or HMDB v3 theoretical fragmentations. To correct for injection order drift, each feature was normalized using data from repeat injections of quality control samples collected every 10 injections throughout the run sequence. Measurement data were smoothed by Locally Weighted Scatterplot Smoothing (LOESS) signal correction (QC-RLSC) as previously described. Values are reported as ratios relative to the median of historical quality control reference samples run with every analytical batch for the given analyte. To account for any potential batch effects, metabolite readouts were median-centered and values were log₁₀-transformed (FIG. 9).

### Assaying of CA19-9

Serum levels of CA19-9 (HCCBP1-58MAG, Millipore, Bedford, MA) were determined by bead-based ELISA assays using Luminex multiplexed assay technology.

### Example 3: Statistical Analysis

Predictive performance estimates for individual microbial-related metabolites identified and quantified through metabolomic profiling of sera were assessed using a receiver operating characteristic curve (ROC). Time-dependent ROC analyses were performed using pROC (version 1.15.3) in the R software environment (version 3.6.1, The R Foundation, r-project.org). The 95% confidence intervals (CI) for AUCs were estimated using the Delong method. Corresponding 95% confidence intervals for odds ratios, adjusted odds ratios, specificity, sensitivity, and the difference measurements were calculated using 1,000 bootstraps. Age, sex, BMI, and smoking status were included as co-variates in the adjusted odds ratio.

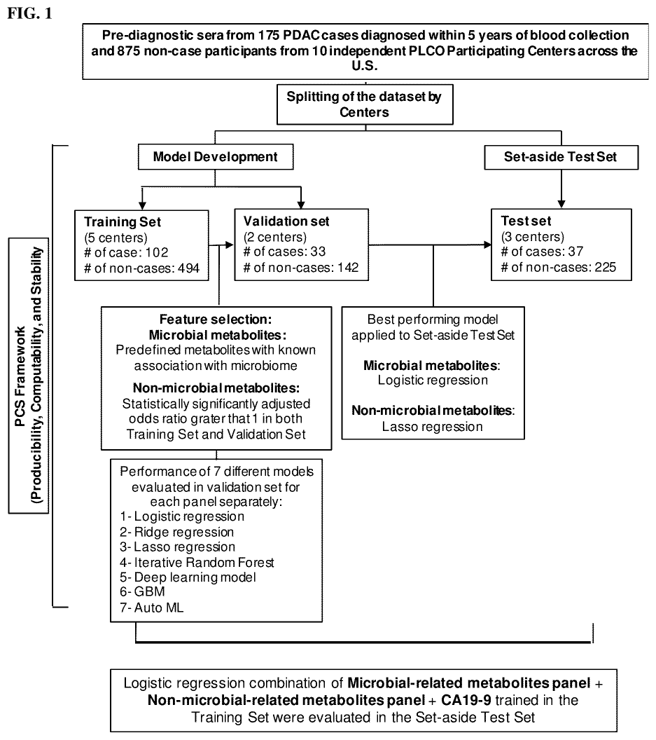

The entire PLCO specimen set was divided into a Development Set that was used for training the models, tuning hyperparameters, and model selection and a set-aside Test Set for obtaining an unbiased evaluation of the selected final model (FIG. 1; Table 3). The Development Set consisted of case and non-case sera from seven of the ten PLCO study centers. The set-aside Test Set consisted of case and non-case sera from the remaining three PLCO study centers.

TABLE 3
Patient characteristics for the Development Set and the Set-Aside Test Set.

| | Development Set | | | | | | | | | | |
|---|---|---|---|---|---|---|---|---|---|---|---|
| | Training Set | | | Validation Set | | | | Set-Aside Test Set | | | |
| | Non-cases | | Cases | Non-cases | | Cases | | Non-cases | | Cases | |
| Total | 494 | | 102 | 142 | | 33 | | 225 | | 37 | |
| Gender, N (%) | | | | | | | | | | | |
| Female | 204 | (41) | 41 (40) | 61 | (43) | 17 | (52) | 91 | (40) | 13 | (35) |
| Male | 290 | (59) | 61 (60) | 81 | (57) | 16 | (48) | 134 | (60) | 24 | (65) |
| Age At Randomization, N | | | | | | | | | | | |
| <=59 | 116 | (23) | 21 (21) | 22 | (15) | 11 | (33) | 45 | (20) | 5 | (14) |
| 60-64 | 108 | (22) | 24 (24) | 34 | (24) | 3 | (9) | 63 | (28) | 14 | (38) |
| 65-69 | 192 | (39) | 41 (40) | 50 | (35) | 9 | (27) | 79 | (35) | 13 | (35) |
| >=70 | 78 | (16) | 16 (16) | 36 | (25) | 10 | (30) | 38 | (17) | 5 | (14) |
| Race, N (%) | | | | | | | | | | | |
| White | 463 | (94) | 99 (97) | 107 | (75) | 24 | (73) | 211 | (94) | 33 | (89) |
| Black | 22 | (4) | 3 (3) | 2 | (1) | 1 | (3) | 6 | (3) | 2 | (5) |
| Other | 9 | (2) | 0 (0) | 33 | (23) | 8 | (24) | 8 | (4) | 2 | (5) |

Seven different learning algorithms were evaluated including deep learning (fully connected feed forward network), gradient boosting machine and auto-machine learning, iterative random forest, LASSO regularization, and logistic regression models. Deep learning, extreme gradient boost, and auto machine learning algorithms were performed in the h2o package in R. Iterative random forests were run using the iRF package in R. To further evaluate model stability in accordance with PCS framework, data perturbations (e.g., via random selection and replacement) were introduced to the Development Set and the performance re-assessed. Based on AUC, a LASSO regression model was selected for subsequent testing in the set-aside Test Set as well as the independent newly diagnosed PDAC cohort.

Samples assayed via metabolomics herein reflect a nested case-control cohort that enriches for cases and, therefore, do not reflect the true risk of pancreatic cancer in the general population. In order to determine the 0.5%, 1%, 1.5% and 2% 5-year risk of pancreatic cancer, a prospective logistic model is estimated from the case-control study that includes an offset term to the logistic model. The offset term is the logit of the prevalence in the population minus the logit of the prevalence in the analyzed dataset. Briefly, absolute risk values for each biomarker were estimated by calculating coefficients of a logistic regression in the training set and the intercept adjusted using the following equation:

$$Risk = \frac{exp ( {β}_{0}^{′} + {β}_{1} \times ( model ) )}{1 + exp ( {β}_{0}^{′} + {β}_{1} \times ( model ) )}$$

$$where$$

$${β}_{0}^{′} = {β}_{0} - log ( \frac{{P}_{data}}{1 - {P}_{data}} ) + log ( \frac{{P}_{Population}}{1 - {P}_{Population}} )$$

In this equation, βo is the intercept derived from logistic regression in the nested case control within a cohort, P_{data} is the prevalence of the disease in the case-enriched dataset, and P_{Population} is the prevalence of the disease in the general population.

For the combination of 3-marker microbial-related metabolite panel (3MMP) and CA19-9, a logistic regression was fitted with the 3MMP and CA19-9 as two separate predictors. The model was developed in the Development Set and validated in the set-aside Test Set.

### Example 4: Microbial-Associated Metabolite Database

To evaluate the association between the microbial-associated metabolites identified in the PLCO specimen sets with distinct microbial species, the Metabolomics Data Explorer database (sonnenburglab.github.io/Metabolomics_Data_Explorer/#/invivo) was used. The database reports the metabolic profiles of 178 gut microorganism strains; microbiota-dependent metabolites were established in diverse biological fluids from gnotobiotic and conventionally colonized mice and traced back to the corresponding metabolomic profiles of cultured bacteria.

Untargeted metabolomics were used to screen for reported microbial-derived metabolites, including short-chain fatty acids, butyrate and acetate, secondary bile acids, indole-derivatives, cadaverine, and TMAO in sera from 175 cases diagnosed within 5 years of blood draw and 875 non-case participants from the PLCO screening trial (Table 1).

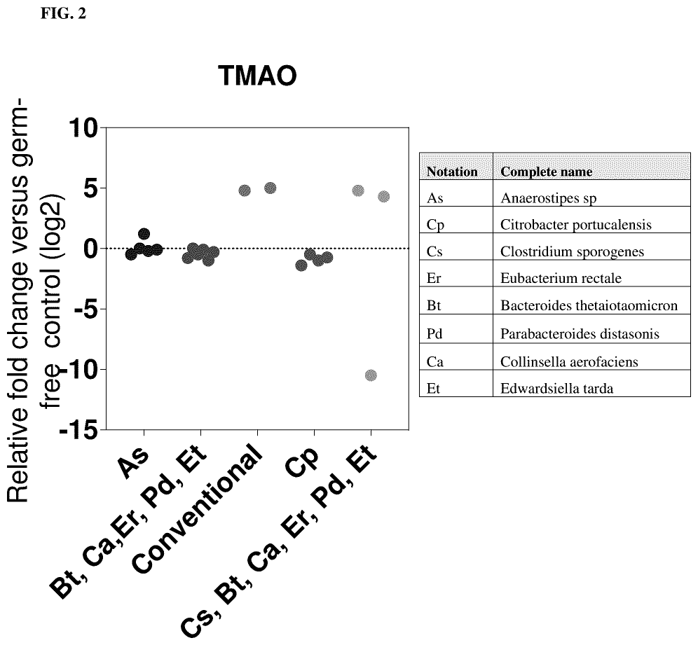

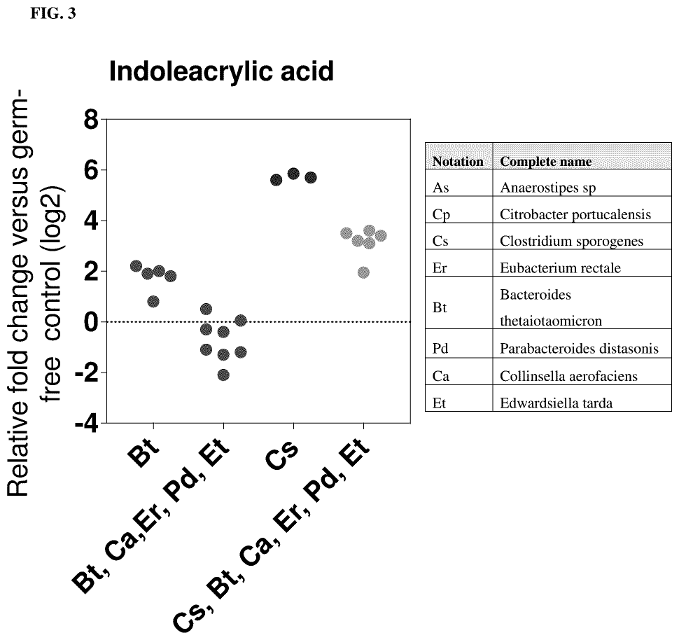

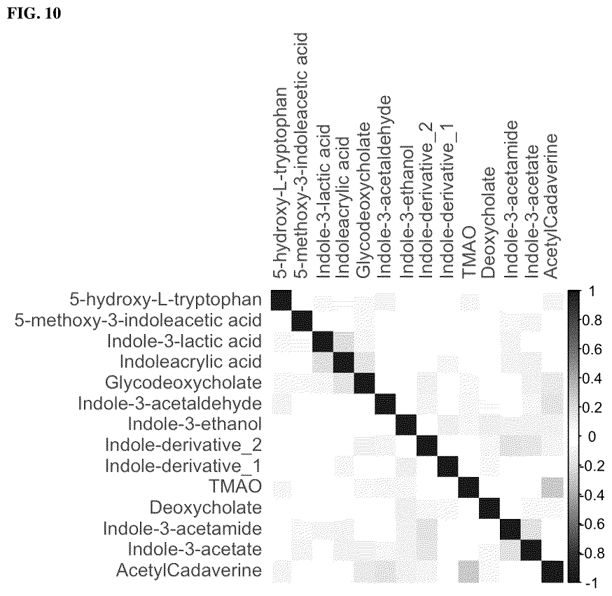

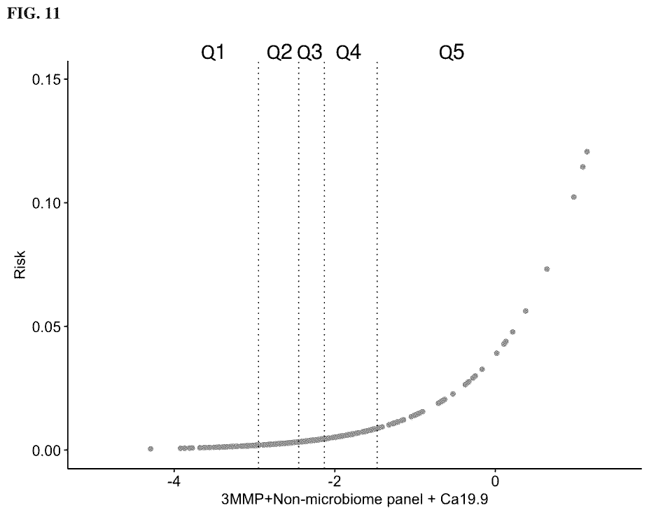

A total of 14 microbial-related metabolites were detected and quantified across all specimens, including nine indole-derivatives, two secondary bile acids, 5-hydroxy-tryptophan, acetylcadaverine, and TMAO. Of the 14 metabolites, indoleacrylic acid, TMAO, and indole-derivative_2 had adjusted ORs per unit standard deviation (SD) increase ≥1.2 for risk of pancreatic cancer (FIGS. 10 and 11). Notably, elevated TMAO and indoleacrylic acid production is associated with phyla of Bacillota, Bacteroidota, Actinomycetota, and Pseudomonadota (species of Clostridium sporogenes (Cs), Eubacterium rectale (Er), Bacteroides thetaiotaomicron (Bt), Parabacteroides distasonis (Pd), Collinsella aerofaciens (Ca), and Edwardsiella tarda (Et)), all of which have relevance to pancreatic cancer (FIGS. 2 and 3).

### Example 5: Model Building and Testing

To establish a combination rule, the predictability, computability, and stability (PCS) framework was applied. All 14 microbial-related metabolites were considered. Seven different models were trained and optimized in the Development Set (FIG. 1). LASSO regression with three selected features (referred to hereon as the 3-marker microbial-related metabolite panel, or 3MMP), including indoleacrylic acid, trimethylamine N-oxide (TMAO), and an indole derivative derived from the metabolism of tryptophan by intestinal microorganisms with an m/z of 177.1032 (indole-derivative_2), achieved the highest performance amongst all models in the Validation Set, yielding an adjusted OR of 1.42 (95% CI 0.94-2.13) per unit SD increase for 5-year probability of pancreatic cancer (Tables 4 and 5). Performance of the 3MMP for risk prediction of pancreatic cancer was not impacted by diabetic status (Table 6). To check scientific reproducibility of the finding, the 3MMP was stress-tested to ensure its reliability on unseen future data. Stable performance across various data perturbations and stability checks demonstrated the robustness and utility of the 3MMP (Table 7).

TABLE 4
Performance of microbial-related metabolites panels in
different learning models in the Validation Set

| Model | Hyperparameters | AUC (95% CI) | Adj OR¹ |
|---|---|---|---|
| Logistic | — | 0.57 (0.46-0.67) | 1.30 (0.85-2.02) |
| regression | | | |
| Ridge | Penalty weight = 0.22 | 0.58 (0.48-0.68) | 1.32 (0.87-2.05) |
| regression | | | |
| Lasso | Penalty weight = 0.023, | 0.64 (0.54-0.73) | 1.42 (0.94-2.13) |
| regression | number of selected | | |
| | features = 3 | | |
| Iterative | Number of iterations = 4 | 0.52 (0.41-0.62) | 1.28 (0.80-1.77) |
| Random | | | |
| Forest | | | |
| Deep | Number of cross-validation | 0.55 (0.45-0.65) | 1.17 (0.75-1.80) |
| learning | folds = 4, hidden layers = 2 | | |
| model | with 64 nodes in each layer | | |
| GBM | Number of trees = 36, | 0.53 (0.41-0.65) | 1.12 (0.76-1.58) |
| | max depth = 6 | | |
| Auto ML | Selected model = | 0.57 (0.45-0.68) | 1.04 (0.64-1.63) |
| | randomized trees | | |

¹Age, sex, BMI, and smoking status were included as covariates in adjusted odds ratios (ORs)

TABLE 5
Selected features and corresponding
model coefficients in LASSO regression.

| | Lasso selection | |
|---|---|---|
| | Selected | |
| Metabolite | in model | Coefficient |
| Acetylcadaverine | — | — |
| 5-hydroxy-L-tryptophan | — | — |
| 5-methoxy-3-indoleacetic acid | — | — |
| Indole-3-lactic acid | — | — |
| Indoleacrylic acid | Yes | 0.3653 |
| Glycodeoxycholate | — | — |
| Indole-3-acetaldehyde | — | — |
| Indole-3-ethanol | — | — |
| Indole-derivative_2 | Yes | 0.5022 |
| Indole-derivative_1 | — | — |
| TMAO | Yes | 0.2412 |
| Deoxycholate | — | — |
| Indole-3-acetamide | — | — |
| Indole-3-acetate | — | — |

TABLE 6
Performance of the 3MMP amongst diabetes population.

| | Diabetes | | Non-Diabetes | |
|---|---|---|---|---|
| | | AUC | | AUC |
| | Numbers | (95% CI) | Numbers | (95% CI) |
| PLCO Testing | N0 = 14 | 0.62 | N0 = 210 | 0.65 |
| Set | N1 = 4 | (0.22-1.00) | N1 = 33 | (0.53-0.77) |
| All PLCO | N0 = 55 | 0.6 | N0 = 805 | 0.62 |
| Samples | N1 =22 | (0.46-0.74) | N1 = 150 | (0.57-0.67) |

TABLE 7
Stability check of the LASSO regression using perturbed
training data and evaluated on the Validation Set.

| | Perturbations | AUC (95% CI) | Adj OR‡ |
|---|---|---|---|
| Lasso | 2 randomly | 0.63 (0.44-0.82) | 1.37 (0.89-2.09) |
| regression | selected centers | | |
| with | 2 randomly | 0.73 (0.60-0.86) | 2.33 (1.52-3.77) |
| 3 selected | selected centers | | |
| features | 2 randomly | 0.54 (0.41-0.68) | 1.25 (0.90-1.73) |
| | selected centers | | |
| | 2 randomly | 0.55 (0.45-0.63) | 1.27 (0.92-1.72) |
| | selected centers | | |
| | 3 randomly | 0.64 (0.54-0.73) | 1.65 (1.23-2.24) |
| | selected centers | | |
| | 300 random | 0.60 (0.51-0.68) | 1.40 (1.02-1.90) |
| | samples | | |

‡Age, sex, BMI, and smoking status were included as covariates in adjusted odds ratios (ORs)

### Performance of the 3MMP in the Independent Newly Diagnosed PDAC Cohort

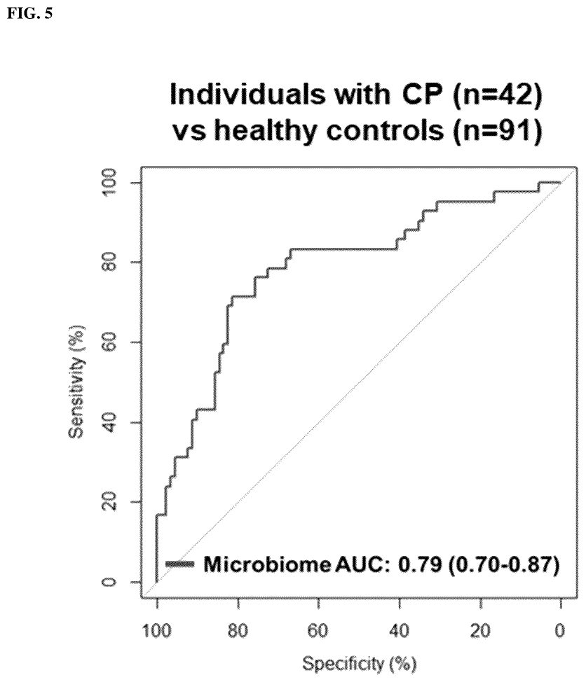

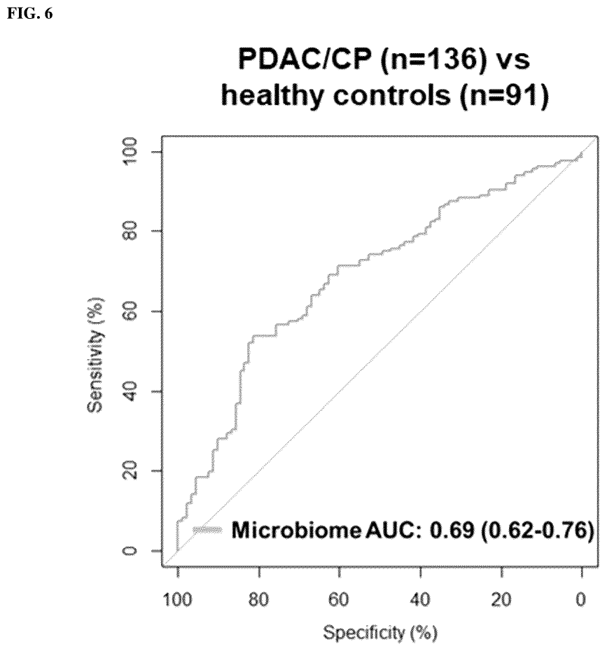

The performance of the 3MMP was further assessed in an independent set of plasma samples from 99 newly diagnosed, resectable PDAC cases, 50 patients with CP, and 100 healthy controls. The 3MMP had an OR of 1.55 (95% CI: 1.13-2.23) per unit SD increase for probability of pancreatic cancer and an OR of 2.07 (95% CI: 1.45-3.18) for pancreatic disease (cancer or chronic pancreatitis) (Table 8). The 3MMP did not differ between PDAC cases and individuals with CP (FIGS. 4-6), highlighting a potential relationship between the microbial-related metabolite panel and inflammation of the pancreas, which is associated with increased cancer risk.

TABLE 8
Odds Ratio (95% CI)

| Resectable PDAC cases | Individuals with CP | PDAC/CP vs |
|---|---|---|
| vs healthy controls | vs healthy controls | healthy controls |
| 1.55 (1.13-2.23) | 2.83 (1.83-4.82) | 2.07 (1.45-3.18) |

Performance of the 3MMP in the set-aside Test Set and the entire PLCO specimen set

In the set-aside Test Set, the 3MMP yielded an AUC of 0.64 (95% CI: 0.53-0.76) and an adjusted OR of 1.72 (95% CI: 1.25-2.37) per unit SD increase for 5-year probability of pancreatic cancer. When considering cases diagnosed within 2 years of blood draw, the 3-marker microbial panel yielded an AUC of 0.61 (95% CI: 0.48-0.74) and an adjusted OR of 1.43 (95% CI: 0.98-2.03) per unit SD increase for risk prediction of pancreatic cancer (Table 9). Performance of the 3-marker microbial panel for risk assessment of pancreatic cancer was similar among diabetic and non-diabetic individuals (Table 10).

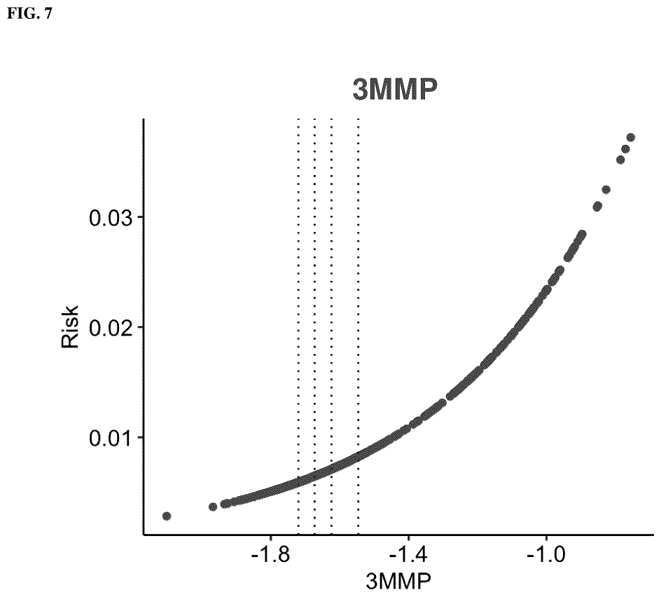

In the entire PLCO specimen set, the 3MMP had an adjusted OR of 1.50 (95% CI: 1.28-1.76) per unit SD increase for 5-year probability of pancreatic cancer (Table 9). The 5-year pancreatic cancer absolute risk estimates for each study participant according to their 3MMP model scores adjusted for prevalence of disease based on the entire intervention arm of the PLCO population are shown in FIG. 7. Absolute 5-year risk estimates for individuals with 3MMP scores in the 80^{th}, 90^{th}, and 95^{th} percentile was 0.83%, 1.44%, and 1.93%, respectively (FIG. 7). At a fixed 1% 5-year risk threshold, the 3MMP had respective sensitivity and specificity of 22.8% (95% CI: 17.4%-29.1%) and 89.1% (95% CI: 87.3%-91.0%) (Table 11).

Combination of the 3MMP with CA19-9 for 5-Year Pancreatic Cancer Risk Assessment

A combined model of the 3MMP+CA19-9 was assessed for improved risk prediction of pancreatic cancer. In the set-aside Test Set, the combined 3MMP+CA19-9 model had an adjusted OR of 3.10 (95% CI: 1.92-5.26) per unit SD increase for 5-year probability of pancreatic cancer, which was improved compared to CA19-9 alone (Adj OR: 2.20 (95% CI: 1.53-3.30)).

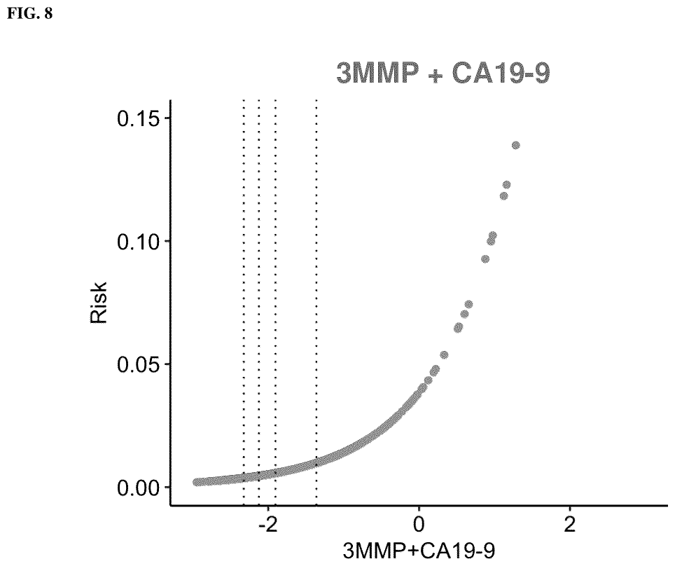

In the entire PLCO specimen set, the combined 3MMP+CA19-9 model had an adjusted OR of 2.69 (95% CI: 2.17-3.37) for 5-year probability of pancreatic cancer (Table 9). Respective 5-year absolute risk estimates for individuals with combined 3MMP+CA19-9 model scores in the 80th, 90th, and 95th percentile was 0.99%, 1.56%, and 2.87% (FIG. 8). The respective sensitivity and specificity of the combined 3MMP+CA19-9 model at the 1% 5-year risk threshold was 46.6% (95% CI: 39.1%-54.3%) and 85.4% (95% CI: 83.1%-87.0%) (Table 12).

TABLE 9
Performance estimates of the 3MMP and a combined 3MMP + CA19-9 model for 5-year risk
prediction of pancreatic cancer in the set-aside Test Set and the entire PLCO specimen set

| | | | 3MMP | | | CA19-9 | | | 3MMP + CA19-9 | | |
|---|---|---|---|---|---|---|---|---|---|---|---|
| Time | Cases, | Non- | AUC | Adj. OR | P- | AUC | Adj. OR | P- | AUC | Adj. OR | P- |
| to Dx | N | cases, N | (95% CI) | (95% CI) | value | (95% CI) | (95% CI) | value | (95% CI) | (95% CI) | value |
| | | | Set-aside Test Set | | | | | | | | |
| [0-5) | 37 | 225 | 0.64 | 1.72 | <0.001 | 0.66 | 2.20 | <0.001 | 0.71 | 3.10 | <0.001 |
| | | | (0.53-0.76) | (1.25-2.37) | | (0.55-0.77) | (1.53-3.30) | | (0.60-0.81) | (1.92-5.26) | |
| [0-2) | 24 | 225 | 0.61 | 1.43 | 0.04 | 0.70 | 2.55 | <0.001 | 0.68 | 3.00 | <0.001 |
| | | | (0.48-0.74) | (0.98-2.03) | | (0.57-0.82) | (1.66-4.19) | | (0.54-0.81) | (1.78-5.54) | |
| [2-5) | 13 | 225 | 0.70 | 2.11 | <0.001 | 0.60 | 1.64 | 0.01 | 0.77 | 3.56 | <0.001 |
| | | | (0.50-0.90) | (1.33-3.43) | | (0.40-0.81) | (0.94-2.89) | | (0.60-0.92) | (1.65-8.27) | |
| | | Entire Set | | | | | | | | | |
| [0-5) | 172 | 861 | 0.62 | 1.50 | <0.001 | 0.68 | 2.27 | <0.001 | 0.71 | 2.69 | <0.001 |
| | | | (0.57-0.67) | (1.28-1.76) | | (0.63-0.73) | (1.89-2.76) | | (0.66-0.75) | (2.17-3.37) | |
| [0-2) | 92 | 861 | 0.60 | 1.43 | <0.001 | 0.75 | 3.21 | <0.001 | 0.76 | 3.77 | <0.001 |
| | | | (0.54-0.67) | (1.18-1.74) | | (0.69-0.81) | (2.50-4.20) | | (0.70-0.82) | (2.83-5.15) | |
| [2-5) | 80 | 861 | 0.64 | 1.53 | <0.001 | 0.60 | 1.48 | <0.001 | 0.64 | 1.81 | <0.001 |
| | | | (0.57-0.70) | (1.28-1.87) | | (0.53-0.67) | (1.18-1.87) | | (0.57-0.70) | (1.39-2.36) | |

TABLE 10
Performance of the 3-marker microbial panel amongst diabetes populations.

| | Diabetics | | | | Non-Diabetic | | | |
|---|---|---|---|---|---|---|---|---|
| | | AUC | Adj. OR | P | | AUC | Adj. OR | P |
| 3MMP | Numbers | (95% CI) | (95% CI) | value | Numbers | (95% CI) | (95% CI) | value |
| PLCO | N0 = 14 | 0.62 | 0.80 | 0.77 | N0 = 210 | 0.64 | 1.84 | <0.001 |
| Testing | N1 = 4 | (0.22-1.00) | (0.09-3.61) | | N1 = 33 | (0.53-0.77) | (1.32-2.61) | |
| Set | | | | | | | | |
| All | N0 = 55 | 0.60 | 1.56 | 0.13 | N0 = 805 | 0.62 | 1.50 | <0.001 |
| PLCO | N1 = 22 | (0.46-0.74) | (0.88-2.95) | | N1 = 150 | (0.57-0.67) | (1.27-1.77) | |
| samples | | | | | | | | |

TABLE 11
Performance estimates of the 3MMP at
0.5%, 1.0%, 1.5%, and 2.0% 5-year
risk thresholds in the set-aside Test Set
and the entire PLCO specimen set.

| | | | Entire PLCO | |
|---|---|---|---|---|
| Risk | Set aside Test Set | | Specimen Set | |
| threshold | Sensitivity | Specificity | Sensitivity | Specificity |
| 0.50% | 0.96 | 0.03 | 0.96 | 0.04 |
| | (0.86-1.00) | (0.01-0.06) | (0.92-0.99) | (0.03-0.05) |
| 1.00% | 0.27 | 0.91 | 0.23 | 0.89 |
| | (0.14-0.41) | (0.87-0.95) | (0.17-0.29) | (0.87-0.91) |
| 1.50% | 0.27 | 0.93 | 0.20 | 0.93 |
| | (0.14-0.41) | (0.89-0.96) | (0.15-0.26) | (0.91-0.94) |
| 2.00% | 0.19 | 0.96 | 0.13 | 0.97 |
| | (0.08-0.32) | (0.94-0.98) | (0.09-0.19) | (0.96-0.98) |

TABLE 12
Performance estimates of the combined 3MMP + CA19-9 model and CA19-9 alone at 0.5%, 1.0%,
1.5%, and 2.0% 5-year risk thresholds in the set-aside Test Set and the entire PLCO specimen set.

| | CA19-9 | | 3MMP + CA19-9 | | Difference (95% CI) | | 1-sided P-value | |
|---|---|---|---|---|---|---|---|---|
| Risk | Sensitivity | Specificity | Sensitivity | Specificity | Sensitivity | Specificity | Sensitivity | Specificity |
| threshold | (95% CI) | (95% CI) | (95% CI) | (95% CI) | (95% CI) | (95% CI) | (95% CI) | (95% CI) |
| | Set-Aside Test Set | | | | | | | |
| 0.50% | 0.79 | 0.36 | 0.69 | 0.55 | −0.08 | 0.19 | 0.211 | <0.001 |
| | (0.65-0.92) | (0.29-0.42) | (0.54-0.85) | (0.48-0.61) | (−0.25-0.04) | (0.12-0.26) | | |
| 1.00% | 0.38 | 0.88 | 0.56 | 0.84 | 0.18 | −0.04 | 0.009 | 0.077 |
| | (0.23-0.55) | (0.83-0.92) | (0.43-0.73) | (0.79-0.89) | (0.06-0.35) | (−0.08-0.00) | | |
| 1.50% | 0.33 | 0.96 | 0.38 | 0.93 | 0.05 | −0.03 | 0.423 | 0.046 |
| | (0.19-0.48) | (0.93-0.98) | (0.21-0.55) | (0.89-0.96) | (−0.06-0.15) | (−0.05-0.00) | | |
| 2.00% | 0.24 | 0.96 | 0.24 | 0.96 | 0 | 0 | 0.961 | 0.785 |
| | (0.1-0.4) | (0.93-0.98) | (0.12-0.39) | (0.93-0.98) | (−0.07-0.09) | (−0.01-0.01) | | |
| | Entire PLCO Specimen Set | | | | | | | |
| 0.50% | 0.79 | 0.38 | 0.69 | 0.52 | −0.10 | 0.14 | 0.003 | <0.001 |
| | (0.73-0.85) | (0.35-0.41) | (0.63-0.77) | (0.5-0.56) | (−0.16-0.03) | (0.11-0.17) | | |
| 1.00% | 0.37 | 0.88 | 0.47 | 0.85 | 0.09 | −0.03 | <0.001 | 0.005 |
| | (0.31-0.44) | (0.86-0.9) | (0.39-0.54) | (0.83-0.87) | (0.05-0.14) | (−0.05-0.01) | | |
| 1.50% | 0.27 | 0.94 | 0.34 | 0.93 | 0.07 | −0.01 | 0.01 | 0.123 |
| | (0.2-0.34) | (0.93-0.96) | (0.27-0.41) | (0.92-0.95) | (0.02-0.13) | (−0.02-0.00) | | |
| 2.00% | 0.22 | 0.97 | 0.28 | 0.97 | 0.06 | 0.00 | 0.005 | 0.983 |
| | (0.16-0.28) | (0.95-0.98) | (0.21-0.35) | (0.95-0.98) | (0.02-0.1) | (−0.01-0.01) | | |

### Contributions of Non-Microbial Metabolites for Improved Risk Prediction of Pancreatic Cancer

The contribution of non-microbial metabolites for pancreatic cancer risk assessment was assessed. A total of 1,009 non-microbial metabolites were quantified in the PLCO specimen set. Five non-microbial metabolites (cholesterol glucuronide, 2-hydroxyglutarate, galactosamine, glucose, and erythritol) exhibited statistically significant adjusted OR in the Development Set (Table 13). The PCS framework was subsequently applied to develop a model based on the five non-microbial metabolites. A logistic regression model was selected based on exhibiting the highest predictive performance in the Validation Set, with a resultant AUC of 0.72 (95% CI: 0.65-0.97) and an adjusted OR of 2.10 (95% CI: 1.04-2.80) for 5-year risk prediction of pancreatic cancer (Tables 14-16). In the set-aside Test Set, the 5-marker non-microbial panel yielded an AUC of 0.74 (95% CI: 0.65-0.83) and an adj OR of 2.72 (95% CI: 1.83-4.24) for 5-year risk prediction of pancreatic cancer.

TABLE 13
Selected features from non-microbial metabolites.

| | Training-5 centers | | Validation-2 centers | |
|---|---|---|---|---|
| | Adj. Odds | Pvalue | Adj. Odds | |
| Name | Ratio | (FDR) | Ratio | Pvalue |
| Cholesterol | 1.735 | <0.001 | 1.720 | 0.006 |
| glucuronide | | | | |
| Galactosamine | 1.749 | <0.001 | 1.514 | 0.035 |
| 2-Hydroxyglutarate | 1.857 | <0.001 | 1.738 | 0.006 |
| Erythritol | 1.688 | <0.001 | 1.532 | 0.030 |
| Glucose | 1.744 | <0.001 | 1.662 | 0.018 |

TABLE 14
Performance of non-microbial-related metabolites panels in
different learning models in the Validation Set.

| Model | Hyperparameters | AUC (95% CI) | Adj OR‡ |
|---|---|---|---|
| Logistic | — | 0.72 (0.65-0.97) | 2.10 (1.04-2.8) |
| regression | | | |
| Ridge | Penalty weight = 0.22 | 0.65 (0.48-0.68) | 1.72 (0.87-2.05) |
| regression | | | |
| Lasso | Penalty weight = 0.023, | 0.69 (0.54-0.73) | 1.92 (0.94-2.13) |
| regression | number of selected | | |
| | features = 3 | | |
| Iterative | Number of iterations = 4 | 0.60 (0.41-0.62) | 1.44 (0.80-1.77) |
| Random | | | |
| Forest | | | |
| Deep | Number of cross-validation | 0.59 (0.45-0.65) | 1.43 (0.75-1.80) |
| learning | folds = 4, hidden layers = 2 | | |
| model | with 64 nodes in each layer | | |
| GBM | Number of trees = 36, | 0.58 (0.41-0.65) | 1.20 (0.76-1.58) |
| | max depth = 6 | | |
| Auto ML | Selected model = | 0.66 (0.45-0.68) | 1.85 (0.64-1.63) |
| | randomized trees | | |

‡Age, sex, BMI, and smoking status were included as covariates in adjusted odds ratios (ORs)

TABLE 15
Selected features and corresponding model
coefficients in logistic regression.

| | Logistic regression |
|---|---|
| Metabolite | coefficient |
| Alpha-D-Glucose | 1.478 |
| Cholesterol glucuronide | 14.941 |
| Galactosamine | 5.415 |
| D-2-Hydroxyglutarate | 4.206 |
| Erythritol | −1.653 |

TABLE 16
Stability check of the 5-marker non-microbial panel using perturbed
training data and evaluated on the Validation Set.

| | Perturbations | AUC (95% CI) | Adj OR‡ |
|---|---|---|---|
| Logistic | 2 randomly selected centers | 0.71 (0.44-0.82) | 2.10 (0.89-2.09) |
| regression | 2 randomly selected centers | 0.73 (0.60-0.86) | 2.33 (1.52-3.77) |
| with 5 | 2 randomly selected centers | 0.69 (0.41-0.68) | 1.87 (0.90-1.73) |
| selected | 2 randomly selected centers | 0.67 (0.45-0.63) | 1.80 (0.92-1.72) |
| features | 3 randomly selected centers | 0.60 (0.54-0.73) | 1.65 (1.23-2.24) |
| | 300 random samples | 0.64 (0.51-0.68) | 1.73 (1.02-1.90) |

‡Age, sex, BMI, and smoking status were included as covariates in adjusted odds ratios (ORs)

To assess the contributions of the 3-marker microbial panel and the 5-marker non-microbial panel, a logistic regression was fitted with the 3-marker microbial panel and the 5-marker non-microbial panel as two separate predictors. The combined metabolite panel yielded an AUC of 0.79 (95% CI: 0.71-0.88) and an adj OR of 3.13 (95% CI: 2.08-4.98) per unit SD increase for 5-year probability of pancreatic cancer in the set-aside Test Set. When considering cases diagnosed within 0-2 years and 2-5 years of blood draw, the combined metabolite panel had respective AUCs of 0.82 (95% CI: 0.72-0.93) and 0.74 (95% CI: 0.60-0.86) (Table 17).

TABLE 17
Performance estimates of the combined 3-marker
microbial panel + 5-marker non-microbial panel for
5-year risk prediction of pancreatic cancer in the set-
aside Test Set and the entire PLCO specimen set.

| | | | | | |
|---|---|---|---|---|---|
| Time to | Cases, | Non-cases, | AUC | Adj. OR† | P- |
| Dx | N | N | (95% CI) | (95% CI) | value |

Set-aside Test Set

| | | | | | |
|---|---|---|---|---|---|
| [0-5) | 37 | 225 | 0.79 (0.71-0.88) | 3.13 (2.08-4.98) | <0.001 |
| [0-2) | 24 | 225 | 0.82 (0.72-0.93) | 3.8 (2.33-6.74) | <0.001 |
| [2-5) | 13 | 225 | 0.74 (0.60-0.86) | 1.90 (1.08-3.37) | 0.02 |

Entire Set

| | | | | | |
|---|---|---|---|---|---|
| [0-5) | 172 | 861 | 0.76 (0.72-0.80) | 2.75 (2.25-3.38) | <0.001 |
| [0-2) | 92 | 861 | 0.81 (0.76-0.86) | 3.66 (2.81-4.84) | <0.001 |
| [2-5) | 80 | 861 | 0.69 (0.63-0.75) | 1.92 (1.51-2.44) | 0.02 |

†Age, sex, BMI and smoking status were included as co-variables in adjusted odd ratios

Contribution of the combined metabolite panel with CA19-9 for pancreatic cancer risk assessment

Levels of CA19-9 have been previously demonstrated to be increased in PDAC cases in the PLCO cohort, with an exponential rise starting two years prior to diagnosis. Therefore, it was assessed whether the combined metabolite panel (3-marker microbial panel plus the 5-marker non-microbial panel) would be complementary with CA19-9 for risk prediction of pancreatic cancer. A logistic regression was fitted with the 3-marker microbial panel, the 5-marker non-microbial panel, and CA19-9 as three separate predictors (Table 18). In the set-aside Test Set, the combined metabolite panel+CA19-9 had an AUC of 0.84 (95% CI: 0.76-0.91) and an adj OR of 9.67 (95% CI: 4.56-23.30) per unit SD increase for 5-year probability of pancreatic cancer (Table 19). For cases diagnosed within 2 years after blood draw, the combined metabolite panel+CA19-9 yielded an AUC of 0.86 (95% CI: 0.77-0.95), which was markedly improved compared to CA19-9 alone (AUC: 0.70 (0.57-0.82), comparison of AUCs p-value: 0.006) (Table 19).

TABLE 18
Selected features and corresponding
model coefficients in logistic regression.

| | Logistic regression |
|---|---|
| Features | coefficient |
| CA19-9 | 0.023 |
| 3-marker | 1.425 |
| microbial panel | |
| 5-marker non- | 0.872 |
| microbial panel | |

TABLE 17
Performance estimates of the CA19.9 and a combined CA19.9 + 3-marker microbial panel + 5-marker non-microbial
panel for 5-year risk prediction of pancreatic cancer in the set-aside Test Set and the entire PLCO specimen set.

| | | | | | CA19.9 + 3-marker microbial panel + | | | Difference | | | |
|---|---|---|---|---|---|---|---|---|---|---|---|
| | | CA19-9 | | | 5-marker non-microbial panel | | | Diff. Of | P value | Diff. of | P value |
| Time | | AUC. | Adj. OR | P | AUC. | Adj. OR | P | AUCs | of Dif- | Adj. OR | of Dif- |
| to Dx | Numbers | (95% CI) | (95% CI)† | value | (95% CI) | (95% CI)† | value | (95% CI) | ference | (95% CI) | ference |

Set-aside Test Set

| | | | | | | | | | | | |
|---|---|---|---|---|---|---|---|---|---|---|---|
| [0-5) | N0 = 225 | 0.66 | 2.2 | <0.001 | 0.84 | 9.67 | <0.001 | 0.18 | <0.001 | 7.47 | 0.003 |
| | N1 = 37 | (0.55-0.77) | (1.53-3.30) | | (0.76-0.91) | (4.56-23.30) | | (0.08-0.25) | | (2.10-15.97) | |
| [0-2) | N0 = 225 | 0.70 | 2.55 | <0.001 | 0.86 | 14.99 | <0.001 | 0.16 | 0.006 | 12.44 | 0.01 |
| | N1 = 24 | (0.57-0.82) | (1.66-4.19) | | (0.77-0.95) | (5.76-47.66) | | (0.05-0.29) | | (2.30-47.40) | |
| [2-5) | N0 = 225 | 0.60 | 1.64 | 0.01 | 0.79 | 5.10 | 0.002 | 0.19 | 0.02 | 3.46 | 0.06 |
| | N1 = 13 | (0.40-0.81) | (0.94-2.89) | | (0.67-0.90) | (1.93-15.88) | | (0.02-0.37) | | (−0.06-13.20) | |

Entire Set

| | | | | | | | | | | | |
|---|---|---|---|---|---|---|---|---|---|---|---|
| [0-5) | N0 = 861 | 0.68 | 2.27 | <0.001 | 0.80 | 8.44 | <0.001 | 0.12 | <0.001 | 6.17 | 0.004 |
| | N1 = 172 | (0.63-0.73) | (1.89-2.76) | | (0.75-0.83) | (5.80-12.20) | | (0.07-0.16) | | (1.80-8.77) | |
| [0-2) | N0 = 861 | 0.75 | 3.21 | <0.001 | 0.87 | 20.02 | <0.001 | 0.12 | <0.001 | 16.81 | <0.001 |
| | N1 = 92 | (0.69-0.81) | (2.50-4.20) | | (0.83-0.91) | (11.51-36.97) | | (0.07-0.16) | | (2.10-27.31) | |
| [2-5) | N0 = 861 | 0.60 | 1.48 | <0.001 | 0.71 | 3.52 | <0.001 | 0.11 | 0.001 | 2.04 | 0.04 |
| | N1 = 80 | (0.53-0.67) | (1.18-1.87) | | (0.65-0.77) | (2.36-5.32) | | (0.57-0.70) | | (1.20-5.86) | |

†Age, sex, BMI and smoking status were included as co-variables in adjusted odd ratios
Log transformation of the values were considered for adjusted odds ratio calculation
N0: number of non-cases
N1: number of cases

### Performance of the Combined Metabolite Panel Plus CA19-9 for 5-Year Risk Assessment of Pancreatic Cancer in the Entire PLCO Specimen Set

In the entire PLCO specimen set, the combined metabolite panels+CA19-9 had an AUC of 0.80 (95% CI: 0.75-0.83) and an adj OR of 8.44 (95% CI: 5.80-12.20) for a 5-year probability of pancreatic cancer and an AUC of 0.87 (95% CI: 0.83-0.91) with an adj OR of 20.02 (95% CI: 11.51-36.97) per unit SD increase for a 2-year probability of pancreatic cancer (Table 17).

The respective 5-year absolute risk estimates, adjusted for prevalence of disease based on the entire intervention arm of the PLCO population for individuals with combined metabolite panel+CA19.9 model scores in the 80^{th}, 90^{th}, and 95^{th} percentile, were 1.07%, 2.05%, and 4.52%, respectively (FIG. 4; Table 19). At a 0.5% risk threshold, the combined metabolite panels+CA19-9 yields a specificity of 0.65 (95% CI:0.62-0.68), sensitivity of 0.87 (95% CI: 0.78-0.94) and cutoff estimate of 1.544. At a 5% risk threshold, the same model yields a specificity of 0.99 (95% CI:0.98-1.00), sensitivity of 0.45 (95% CI: 0.33-0.58) and cutoff estimate of 3.367.

TABLE 19
Absolute 5-year risk estimates for individuals
with CA19-9 alone and CA19-9 + 3-marker
microbial panel + 5-marker non-microbial
panel scores.

| | 5-year absolute risk (%) | |
|---|---|---|
| Percentile | | CA19-9 + |
| values | CA19-9 | metabolite panels |
| 20.0% | 0.450 | 0.227 |
| 40.0% | 0.517 | 0.350 |
| 60.0% | 0.609 | 0.528 |
| 80.0% | 0.870 | 1.066 |
| 90.0% | 1.389 | 2.049 |
| 95.0% | 2.159 | 4.521 |
| 97.5% | 10.060 | 13.330 |

### Performance Estimates of the Metabolite (Host- and Microbial-Derived) Panel Based on PEB or ST Methods for Detection of PDAC

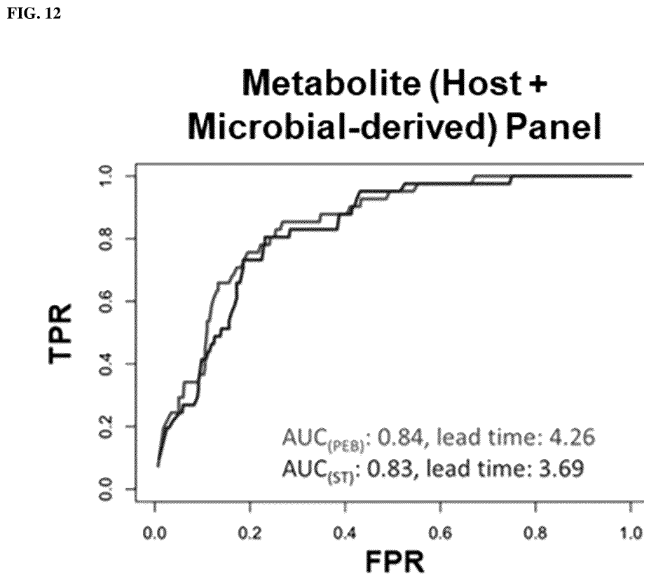

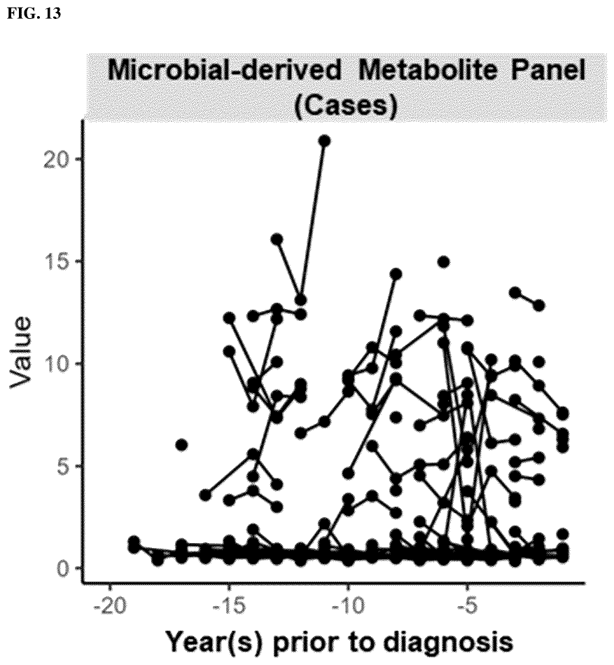

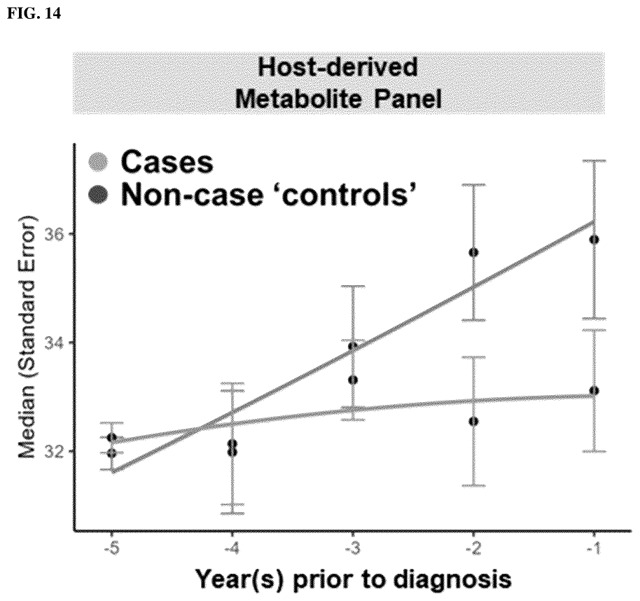

The reliability of the metabolite (host+microbial-derived) panel for risk assessment of PDAC was next assessed. Here, pre-diagnostic plasmas from 242 PDAC cases and 242 matched non-case ‘control’ participants were leveraged from the PLCO cohort. Controls were matched to cases based on age, sex, race, and date of PDAC diagnosis. The specimen set includes serial samples (n=up to 3) collected from both cases and non-cases. The parametric empirical Bayes (PEB) longitudinal algorithm, which incorporates participant biomarker history at each test, and the single-threshold (ST), which only consider single time point value, approaches were applied and assessed using ROC curve characteristics. For these analyses, analyses were restricted to participants with at least two biomarker measurements and, for case participants, at least one biomarker measurement within 3 years of a PDAC diagnosis. In total, 41 PDAC cases and 168 non-cases were selected, of which 18 cases and 68 controls had at least 2 blood draws and 23 cases and 100 non-cases had 3 blood draws. Predictive performance estimates of the PEB and ST method for the metabolite panel were similar, yielding AUC estimates of 0.84 and 0.83, respectively. At the 2% FPR, the PEB algorithm signaled an average of 4.26 years prior to diagnosis whereas the ST method signaled an average of 3.69 years before diagnosis (FIG. 12). Cases consistently had elevated levels of microbial-metabolites regardless of time from blood draw to clinical diagnosis compared to non-cases (FIG. 13), whereas host-derived metabolites exhibited initial increases starting at three years prior to diagnosis (FIG. 14).

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of treatment of pancreatic ductal adenocarcinoma (PDAC) in a patient having an elevated risk score or positive risk profile based on the patient's measured levels of trimethylamine N-oxide (TMAO), indoleacrylic acid, and an indole derivative derived from the metabolism of tryptophan by intestinal microorganisms, and optionally, levels of cholesterol glucuronide, 2-hydroxyglutarate, galactosamine, glucose, erythritol, and CA19-9, wherein the elevated risk score or positive risk profile led to the patient's diagnosis with PDAC, comprising administering a therapeutically effective amount of a treatment for PDAC to the patient.

2. A method of treatment of pancreatic ductal adenocarcinoma (PDAC), comprising:
a) identifying a patient with an elevated risk score or positive risk profile based on the patient's measured levels of trimethylamine N-oxide (TMAO), indoleacrylic acid, and an indole derivative derived from the metabolism of tryptophan by intestinal microorganisms, and optionally, levels of cholesterol glucuronide, 2-hydroxyglutarate, galactosamine, glucose, erythritol, and CA19-9, wherein the elevated risk score or positive risk profile led to the patient's diagnosis with PDAC; and
b) administering a therapeutically effective amount of a treatment for PDAC to the patient.

3. A method of determining the risk of a subject for pancreatic ductal adenocarcinoma (PDAC), comprising, in a biological sample obtained from the subject:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in the biological sample; and
b) classifying the subject as being at risk of PDAC or not at risk of PDAC based on the measured levels.

4. A method of producing a risk profile of a subject for pancreatic ductal adenocarcinoma (PDAC), comprising, in a biological sample obtained from the subject:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in the biological sample; and
b) classifying the risk profile of the subject as being at risk of PDAC (positive) or not at risk of PDAC (negative) based on the measured levels.

5. A method for calculating a patient's biomarker scores or risk score for PDAC, comprising:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in a biological sample obtained from the patient; and
b) calculating the biomarker scores or risk score using the numerical values of the measured levels in a machine learning model.

6. A method of risk stratification for a patient at risk for PDAC, comprising, in a biological sample obtained from the patient:
a) measuring the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, in the biological sample; and
b) determining, by processor circuitry, the risk score for the patient, wherein the risk score is determined via a scoring function derived from metabolite profiles for biological samples taken from a plurality of individuals that were monitored for PDAC.

7. The method of claim 5, wherein the machine learning model is a logistic regression.

8. The method of claim 5, wherein the machine learning model is a LASSO regularization.

9. The method of either claim 5 or 6, wherein the biomarker scores or risk score for PDAC are/is calculated with the equation: 0.3653*[indoleacrylic acid]+0.2412*[TMAO]+0.5022*[indole derivative].

10. The method of either claim 5 or 6, wherein the biomarker scores or risk score for PDAC are/is calculated with the equation: 1.478*[alpha-D-glucose]+14.941*[cholesterol glucuronide]+5.415*[galactosamine]+4.206*[D-2-hydroxyglutarate]+—1.653*[erythritol].

11. The method of either claim 5 or 6, wherein the biomarker scores or risk score for PDAC are/is calculated with the equation: 0.023*[CA19-9]+1.425*(0.3653*[indoleacrylic acid]+0.2412*[TMAO]+0.5022*[indole derivative])+0.872*(1.478*[alpha-D-glucose]+14.941*[cholesterol glucuronide]+5.415*[galactosamine]+4.206*[D-2-hydroxyglutarate]+—1.653*[erythritol]).

12. The method as recited in any of claims 1-6, further comprising measuring the levels of or identifying a patient with elevated levels of cholesterol glucuronide, 2-hydroxyglutarate, galactosamine, glucose, and erythritol.

13. The method as recited in any of claims 1-6, further comprising measuring the level of CA19-9.

14. The method as recited in any of claims 1-6, wherein the indole derivative has a molecular weight of less than 500 daltons.

15. The method as recited in claim 14, wherein the indole derivative is a compound of structural Formula I: 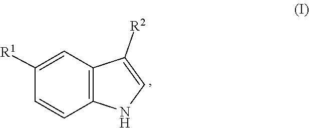
wherein:
R¹ is chosen from hydrogen, hydroxy, and C₁₋₄alkoxy, any of which may be optionally substituted;
R² is chosen from hydrogen, 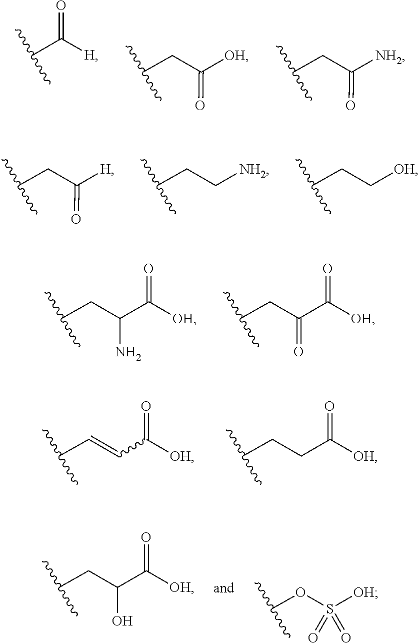
and
the compound of Formula I is not chosen from 5-hydroxy-L-tryptophan, 5-methoxy-3-indoleacetic acid, indole-3-lactic acid, indole-3-acetaldehyde, indole-3-ethanol, indole-3-acetamide, and indole-3-acetate.

16. The method of claim 14 or 15, wherein the indole derivative has a molecular weight of about 177 daltons.

17. The method as recited in any of claims 1-16, wherein the risk score based on the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, is elevated relative to a reference patient or group that does not have PDAC.

18. The method as recited in any of claims 1-16, wherein the risk score based on the levels of trimethylamine N-oxide (TMAO), indoleacrylic acid, and an indole derivative derived from the metabolism of tryptophan by intestinal microorganisms, and optionally, the levels of cholesterol glucuronide, 2-hydroxyglutarate, galactosamine, glucose, erythritol, and CA19-9, is elevated relative to a reference patient or group that has chronic pancreatitis or benign pancreatic disease.

19. The method as recited in any of claims 1-16, wherein the PDAC is diagnosed at or before the borderline respectable stage or at the respectable stage.

20. The method as recited in any of claims 1-16, wherein the patient is over 50 years old.

21. The method as recited in any of claims 1-16, wherein the patient has new-onset diabetes mellitus, or an asymptomatic variant thereof.

22. The method as recited in any of claims 1-16, wherein the patient has chronic pancreatitis, or an asymptomatic variant thereof.

23. The method as recited in any of claims 1-16, wherein the patient has been incidentally diagnosed with mucin-secreting cysts of the pancreas, or an asymptomatic variant thereof.

24. The method of any preceding claim, wherein each of trimethylamine N-oxide (TMAO), indoleacrylic acid, and an indole derivative derived from the metabolism of tryptophan by intestinal microorganisms, and optionally, each of cholesterol glucuronide, 2-hydroxyglutarate, galactosamine, glucose, erythritol, and CA19-9, generates a detectable signal.

25. The method of claim 24, wherein the detectable signals are detectable by a spectrometric method.

26. The method of claim 25, wherein the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

27. The method of claim 26, wherein the spectrometric method is mass spectrometry.

28. The method of claim 27, wherein the mass spectrometry is LC-TOF-MS.

29. The method of claim 1, wherein the treatment is chosen from surgery, chemotherapy, immunotherapy, radiation therapy, targeted therapy, or a combination thereof.

30. The method of any of claims 1-6, wherein the calculated biomarker scores, risk score, or risk profile are/is based on sensitivity and specificity values that corresponds to the risk threshold of the subject for PDAC.

31. The method of claim 30, wherein the risk profile has sensitivity and specificity values that do not differ substantially from the curve in FIG. 4.

32. The method of claim 31, wherein the sensitivity and specificity values differ by less than 10%.

33. The method of claim 32, wherein the sensitivity and specificity values differ by less than 5%.

34. The method of claim 33, wherein the sensitivity and specificity values differ by less than 1%.

35. The method of any of claims 1-6, wherein the cutoff point comprises an AUC (95% CI) of at least 0.57.

36. The method of claim 35, wherein the AUC of the method is greater than 0.77.

37. The method of claim 36, wherein the AUC of the method is between 0.77 and 0.95.

38. The method of claim 37, wherein the AUC of the method is about 0.86.

39. The method of claim 38, wherein the AUC of the method is 0.86.

40. The method of claim 37, wherein the AUC of the method is about 0.84.

41. The method of claim 40, wherein the AUC of the method is 0.84.

42. The method of claim 37, wherein the AUC of the method is about 0.79.

43. The method of claim 42, wherein the AUC of the method is 0.79.

44. The method of any of claims 35-43, wherein the AUC of the method is greater than the AUC for a different biomarker, biomarkers, panel, assay, algorithm, model, or any combination thereof.

45. The method of claim 44, wherein the biomarker is CA19-9 alone.

46. The method of claim 45, wherein the cutoff points of the respective AUCs are used for classification.

47. The method of claim 45, analyzed by the same statistical methods.

48. The method of any of claims 1-6, wherein the 5-year odds ratio (OR) for the probability of developing PDAC is between 1.13 and 2.23.

49. The method of claim 48, wherein the OR is between 1.3 and 1.8.

50. The method of claim 49, wherein the OR is about 1.55.

51. The method of claim 50, wherein the OR is 1.55.

52. The method as recited in any previous claim, further comprising assigning the patient to an appropriate risk group based on the calculated risk score.

53. The method of claim 52, wherein there are at least two risk groups.

54. The method of any of claims 1-6, wherein the adjusted odds ratio (AOR) for the probability of developing PDAC per unit standard deviation (SD) is between 0.98 and 47.66.

55. The method of claim 54, wherein the AOR is between 1.5 and 10.0.

56. The method of claim 55, wherein the AOR is about 1.72.

57. The method of claim 56, wherein the AOR is 1.72.

58. The method of claim 55, wherein the AOR is about 3.13.

59. The method of claim 58, wherein the AOR is 3.13.

60. The method of claim 55, wherein the AOR is about 9.67.

61. The method of claim 60, wherein the AOR is 9.67.

62. The method of any of claims 55-61, wherein the AOR represents the odds of developing PDAC within the next 5 years.

63. The method of claim 54, wherein the AOR is between 1.4 and 15.0.

64. The method of claim 63, wherein the AOR is about 1.43.

65. The method of claim 64, wherein the AOR is 1.43.

66. The method of claim 63, wherein the AOR is about 3.8.

67. The method of claim 66, wherein the AOR is 3.8.

68. The method of claim 63, wherein the AOR is about 14.99.

69. The method of claim 68, wherein the AOR is 14.99.

70. The method of any of claims 63-69, wherein the AOR represents the odds of developing PDAC within the next 2 years.

71. The method of claim 54, wherein the AOR is between 1.5 and 6.0.

72. The method of claim 71, wherein the AOR is about 2.11.

73. The method of claim 72, wherein the AOR is 2.11.

74. The method of claim 71, wherein the AOR is about 1.90.

75. The method of claim 74, wherein the AOR is 1.90.

76. The method of claim 71, wherein the AOR is about 5.10.

77. The method of claim 76, wherein the AOR is 5.10.

78. The method of any of claims 71-77, wherein the AOR represents the odds of developing PDAC within the next 2 to 5 years.

79. The method of any of claims 54-78, wherein the AOR value is controlled for age, sex, smoking and body mass index (BMI).

80. The method of claim 79, wherein the AOR value is additionally controlled for diabetic status.

81. The method as recited in any previous claim, wherein the risk score is measured against a given threshold value that represents the absolute risk of developing PDAC over the next five years.

82. The method of claim 81, wherein the threshold value is greater than 0.001, or 0.1%.

83. The method of claim 82, wherein the threshold value is between 0.005 and 0.1, or 0.5% and 10%.

84. The method of claim 83, wherein the threshold value is about 0.01, or 1%.

85. The method of claim 84, wherein the threshold value is 0.01, or 1%.

86. The method of any of claims 81-85, wherein the risk score exceeds the threshold value and the patient is classified as being at risk for PDAC.

87. The method of any of claims 81-85, wherein the risk score is below the threshold value and the patient is classified as being not at risk for PDAC.

88. The method of claim 86, wherein the patient is subsequently designated for pancreatic cancer screening.

89. The method of claim 88, wherein the screening is chosen from endoscopic ultrasound, magnetic resonance imaging (MRI), and computed topography (CT) scans.

90. The method of claim 88, wherein the screening is performed annually.

91. The method of claim 88, wherein the screening is performed semi-annually.

92. The method as recited in any one of claims 1-6, further comprising measuring the levels of trimethylamine N-oxide (TMAO), indoleacrylic acid, and/or an indole derivative derived from the metabolism of tryptophan by intestinal microorganisms, and optionally, levels of cholesterol glucuronide, 2-hydroxyglutarate, galactosamine, glucose, erythritol, and CA19-9, in at least one or more additional biological samples obtained from the patient and classifying the patient with an elevated risk score or positive risk profile OR being at risk or not at risk of PDAC.

93. The method of claim 92, wherein the patient classification is calculated in a machine learning model.

94. The method of claim 93, wherein the machine learning model is a parametric empirical Bayes longitudinal algorithm.
